(19) United States
(12) Patent Application Publication
(10) Pub. No.: US 2025/0177763 A1
(43) Pub. Date: Jun. 5, 2025

(54) BATTERY LOCKING MECHANISMS FOR A WEARABLE MEDICAL DEVICE

(71) Applicant: ZOLL Medical Corporation, Chelmsford, MA (US)

(72) Inventors: Phillip H. Amsler, Cheswick, PA (US); Nicholas J. Chernansky, Oakmont, PA (US); Andrew J. Malkiewicz, Pittsburgh, PA (US); Sunaina Rustagi, Presto, PA (US)

(21) Appl. No.: 18/727,627

(22) PCT Filed: Jan. 9, 2023
(86) PCT No.: PCT/US2023/010392

§ 371 Date: Jul. 9, 2024

Related U.S. Application Data
(60) Provisional application No. 63/298,020, filed on Jan. 10, 2022.

(51) Int. Cl. A61N 1/39

(52) U.S. Cl. CPC A61N 1/3975; A61N 1/3968; A61N 1/3993

(57) ABSTRACT

A wearable cardioversion-defibrillation system for providing battery information is provided. The system includes a controller operably coupled to one or more sensing electrodes and one or more therapy electrodes, a battery well disposed on the controller, and battery circuitry disposed in either a removable battery or within the battery well. The battery circuitry can be configured to detect whether the removable battery is inserted into the battery well and providing power to monitor and/or treat the patient, and provide human-perceptible confirmation via one or more of tactile, visual, or audio feedback to the patient on detecting that the removable battery has been inserted into the battery well and is providing power to monitor and/or treat the patient. Upon proper insertion of the removable battery within the battery well, the controller can be configured to monitor and/or treat the patient for a cardiac arrhythmia based on power from the removable battery.

### BACKGROUND

The present disclosure is directed to providing an indication to a patient that a power source is secured to provide power to an ambulatory medical device.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk, have been hospitalized for, or otherwise are suffering from, adverse heart conditions can be prescribed a wearable cardiac monitoring and/or treatment device. In addition to the wearable device, the patient can also be given a battery charger and a set of rechargeable batteries. As the wearable device is generally prescribed for continuous or near-continuous use (e.g., only to be removed when bathing), the patient is generally instructed to keep a battery in the device at all times and one battery on the charger at all times. Thus, as one battery is being depleted by the device, the second battery is being charged. By following these instructions, when a battery swap is required, the second battery is charged and ready to power the wearable device. Upon swapping, the battery removed from the wearable device is inserted into the charger, and the process is repeated.

### SUMMARY

In an example, a wearable cardioversion-defibrillation system for providing battery information to patients is provided. The system includes one or more sensing electrodes configured to output a signal indicative of cardiac activity of a patient, one or more therapy electrodes configured to provide one or more treatment shocks to the patient when a patient wearing the wearable cardioversion-defibrillation system experiences a cardiac arrhythmia, a controller of the wearable cardioversion-defibrillation system, the controller operably coupled to the one or more sensing electrodes and the one or more therapy electrodes, a battery well disposed on the controller; and battery circuitry disposed in either a removable battery or within the battery well. The battery circuitry is configured to detect whether the removable battery is inserted into the battery well and providing power to monitor and/or treat the patient, and provide human-perceptible confirmation via one or more of tactile, visual, or audio feedback to the patient on detecting that the removable battery has been inserted into the battery well and is providing power to monitor and/or treat the patient, wherein upon proper insertion of the removable battery within the battery well, the controller is configured to monitor and/or treat the patient for a cardiac arrhythmia based on power from the removable battery.

Implementations of the wearable cardioversion-defibrillation system for providing battery information to patients can include one or more of the following features.

In examples of the wearable cardioversion-defibrillation system, the battery circuitry can include at least one feedback device configured to output the one or more tactile, visual, or audio feedback. In some examples, the at least one feedback device can include at least one visual indicator configured to transition from a first visual state to a second visual state upon proper insertion of the removable battery into the battery well. In some examples, the at least one feedback device can include at least one audio output device configured to output the audio feedback upon proper insertion of the removable battery into the battery well. In some examples, the at least one audio output device can be adjustable to alter a predetermined frequency range of the audio feedback. In some examples, the at least one audio output device can be adjustable via a control provided during initial patient fitting of the wearable cardioversion-defibrillation system. In some examples, the at least one feedback device can include a vibrational mechanism configured to output the tactile feedback when the removable battery is inserted into the battery well. In some examples, the removable battery can include a housing and the at least one feedback device is integrated into the housing of the removable battery.

In examples of the wearable cardioversion-defibrillation system, the battery well can include an electrical connector configured to receive at least a portion of the removable battery to establish an electrical connection between the removable battery and the controller. In some examples, the battery well can further include a seal configured to at least partially deform when the removable battery is inserted into the battery well to securely enclose the electrical connection between the removable battery and the controller.

In examples of the wearable cardioversion-defibrillation system, the system can further include a battery charger, the battery charger including a charging battery well, the charging battery well including a charging connector. In some examples, the battery circuitry can be disposed in the removable battery and includes at least one additional feedback device configured to provide one or more of the tactile, visual, or audio feedback to the patient when the removable battery is inserted into the charging battery well and a charging electrical connection is established between the removable battery and the charging connector.

In examples of the wearable cardioversion-defibrillation system, the system can further include a battery latching mechanism configured to secure the removable battery in the battery well and provide additional tactile feedback to the patient upon proper insertion of the removable battery within the battery well.

In examples of the wearable cardioversion-defibrillation system, the battery well can include a spring mechanism configured to exert a spring force opposing insertion of the removable battery into the battery well. In some examples, the system can further include a battery latching mechanism configured to secure the removable battery in the battery well and oppose the spring force such that, upon release of the battery latching mechanism, the spring force is configured to assist in removal of the removable battery from the battery well. In some examples, the battery latching mechanism can be further configured to provide additional tactile feedback to the patient upon proper insertion of the removable battery within the battery well.

In examples of the wearable cardioversion-defibrillation system, the battery circuitry can further include battery detection circuitry configured to detect a position of the removable battery within the battery well. In some examples, the battery detection circuitry can include one or more of a Hall effect sensor, an optical position sensor, a mechanical switch, an infrared position sensor, and an electrical connector.

In examples of the wearable cardioversion-defibrillation system, the removable battery can be configured to provide power to the one or more therapy electrodes to deliver the one or more treatment shocks to the patient.

In examples of the wearable cardioversion-defibrillation system, the removable battery can be configured to provide power to the controller to monitor the signal indicative of cardiac activity of the patient for a cardiac arrhythmia.

In another example, a system for providing battery insertion feedback to patients wearing or using a wearable cardioversion-defibrillation device is provided. The system includes a controller of the wearable cardioversion-defibrillation device, a battery well disposed on the controller, a removable battery configured to be inserted into the battery well such that an electrical connection is established between the removable battery and the controller, a mechanical attachment disposed on either the removable battery or within an interior volume of the battery well, the mechanical attachment configured at least to detect the insertion of the removable battery within the battery well, and a visual indicator operably coupled to the mechanical attachment, the visual indicator configured to provide visual feedback to a patient using the wearable cardioversion-defibrillation device that the removable battery is inserted into the battery well and providing power to the wearable cardioversion-defibrillation device to monitor and/or treat the patient.

Implementations of the system for providing battery insertion feedback to patients wearing or using a wearable cardioversion-defibrillation device can include one or more of the following features.

In examples of the system, the mechanical attachment can be further configured to exert an opposition force upon the removable battery as the removable battery is inserted within the battery well. In some examples, the mechanical attachment can be further configured such that, upon release of the removable battery from the battery well, the opposition force causes at least partial ejection of the removable battery from the battery well. In some examples, the visual indicator can be configured to alter the visual feedback in response to changes in the opposition force exerted by the mechanical attachment as the removable battery is inserted within the battery well.

In examples of the system, the mechanical attachment can include at least one movable pawl operably coupled to the visual indicator, the movable pawl configured to be displaced from within an interior volume of the battery well as the removable battery is inserted into the battery well. In some examples, the at least one movable pawl can be shaped such that insertion of the removable battery into the battery causes displacement of at least a portion of the movable pawl. In some examples, movement of the movable pawl can causes movement of at least a portion of the visual indicator, thereby changing the visual feedback provided to the patient.

In examples of the system, the battery well can include an electrical connector configured to receive at least a portion of the removable battery to establish the electrical connection between the removable battery and the controller. In some examples, the battery well can further include a seal configured to at least partially deform when the removable battery is inserted into the battery well to securely enclose the electrical connection between the removable battery and the controller.

In examples of the system, the removable battery can include a housing and the visual indicator is integrated into the housing of the removable battery.

In examples of the system, the system can further include a battery charger, the battery charger including a charging battery well, the charging battery well including a charging connector.

In examples of the system, the system can further include a battery latching mechanism configured to secure the removable battery in the battery well and provide tactile feedback to the patient upon proper insertion of the removable battery within the battery well.

In examples of the system, the battery well can include a spring mechanism configured to exert a spring force opposing insertion of the removable battery into the battery well.

In some examples, the system can include a battery latching mechanism configured to secure the removable battery in the battery well and oppose the spring force such that, upon release of the battery latching mechanism, the spring force is configured to assist in removal of the removable battery from the battery well. In some examples, the battery latching mechanism can be further configured to provide additional tactile feedback to the patient upon proper insertion of the removable battery within the battery well.

In examples of the system, the system can further include battery circuitry disposed in either the removable battery or within the battery well, the battery circuitry configured to detect whether the removable battery is inserted into the battery well and providing power to monitor and/or treat the patient. In some examples, the battery circuitry can include at least one feedback device configured to output the one or more tactile, visual, or audio feedback. In some examples, the at least one feedback device can include at least one audio output device configured to output the audio feedback upon proper insertion of the removable battery into the battery well. In some examples, the at least one audio output device can be adjustable to alter a predetermined frequency range of the audio feedback. In some examples, the at least one audio output device can be adjustable via a control provided during initial patient fitting of the wearable cardioversion-defibrillation system. In some examples, the at least one feedback device can include a vibrational mechanism configured to output the tactile feedback when the removable battery is inserted into the battery well. In some examples, the battery circuitry can further include battery detection circuitry configured to detect a position of the removable battery within the battery well. In some examples, the battery detection circuitry can include one or more of a Hall effect sensor, an optical position sensor, a mechanical switch, an infrared position sensor, and an electrical connector.

In examples of the system, the removable battery can be configured to provide power to one or more therapy electrodes the wearable cardioversion-defibrillation device to deliver one or more treatment shocks to the patient.

In examples of the system, the removable battery can be configured to provide power to a controller the wearable cardioversion-defibrillation device to monitor a signal indicative of cardiac activity of the patient for a cardiac arrhythmia.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 illustrates a schematic view of a sample controller for a wearable medical device, in accordance with an example of the present disclosure.

FIG. 2 illustrates a schematic view of a rechargeable battery including battery circuitry and a feedback mechanism, in accordance with an example of the present disclosure.

FIGS. 3A and 3B illustrates schematic views of a battery charger for a rechargeable battery for a wearable medical device, in accordance with an example of the present disclosure.

FIGS. 4A-4F illustrate schematic views of various examples of battery circuitry, in accordance with various examples of the present disclosure.

FIG. 5 illustrates a process flow for monitoring battery insertion and providing insertion feedback, in accordance with an example of the present disclosure.

FIGS. 6A and 6B illustrate examples of a visual indicator for providing insertion feedback, in accordance with an example of the present disclosure.

FIGS. 6C and 6D illustrate additional examples of a rechargeable battery including a visual indicator, in accordance with an example of the present disclosure.

FIGS. 7A and 7B illustrate examples of a mechanical attachment including movable pawls, in accordance with an example of the present disclosure.

FIGS. 7C and 7D illustrate additional examples of a rechargeable battery including movable pawls, in accordance with an example of the present disclosure

FIGS. 8A and 8B illustrate examples of a mechanical attachment including angled receptacles for receiving one or more movable pawls, in accordance with an example of the present disclosure.

FIGS. 9A and 9B illustrate examples of a mechanical attachment including partial latch springs, in accordance with an example of the present disclosure.

FIGS. 10A and 10B illustrate examples of an optical feedback mechanism, in accordance with an example of the present disclosure.

FIGS. 11A and 11B illustrate examples of an electrical connector mechanism, in accordance with an example of the present disclosure

FIG. 12 illustrates an example of a battery position sensor, in accordance with an example of the present disclosure

FIG. 13 illustrates an example of an ejection spring, in accordance with an example of the present disclosure.

FIG. 14 illustrates an example of a battery connector seal, in accordance with an example of the present disclosure

FIGS. 15A-15D depict sample ambulatory medical devices that may be prescribed to a heart failure patient, in accordance with an example of the present disclosure.

FIG. 16 illustrates an example battery insertion process as used during an example study of the battery mechanisms as described herein.

FIGS. 17-20 illustrate results graphs from the example study of the battery mechanisms as described herein.

### DETAILED DESCRIPTION

Wearable medical devices, such as cardiac event monitoring and/or treatment devices, are used in clinical or outpatient settings to monitor and/or record various ECG and other physiological signals for a patient. These ECG and other physiological signals can be used to determine a current condition for a patient as well as to provide an indication that the patient may require treatment such as a defibrillation shock.

Wearable medical devices are powered by either an integrated rechargeable battery or by a removable rechargeable battery. In some scenarios, where a patient is required to wear a wearable medical device, such as a wearable cardioverter defibrillator (WCD) for multiple hours in a day, e.g., a majority of the hours of the day, and only to removed when bathing, it may not be practical to prescribe a device that includes an integrated rechargeable battery. In such scenarios, the patient can be given two rechargeable batteries and guided to insert one battery in the medical device, to insert the second battery into the charger, and to swap the batteries when the remaining runtime of the battery in the medical device drops below a predetermined threshold value. When the batteries are to be swapped, the first battery is removed from the WCD and temporarily placed aside. The second battery is removed from the charger and inserted into the WCD. Once the WCD has restarted and/or power to the device is resumed, such that the WCD is being properly powered by the second battery, the patient places the first battery onto the charger for charging. By following this guidance, when a battery swap is required (e.g., every 24 hours), a fully charged battery is sitting in the charger and ready to power the WCD.

Example systems and methods disclosed here are advantageous in that they can mitigate potential risks in the event the guidance noted above in connection with battery maintenance for WCDs is not followed. As an example of potential risk, if the patient improperly places a depleted battery onto the charger, when time for the next battery swap arrives, the patient may have a compromised battery (e.g., not completely charged battery or only partially charged) to insert into the medical device. Such a scenario can result in the medical device not functioning appropriately (e.g., wherein substantially all of the safety critical functions are operational) or at all (e.g., the device may not power on at all). The systems and methods disclosed herein, however, advantageously mitigate these concerns by providing visual and/or other feedback to a user alerting them to the situation so that corrective action may be taken. For example, with a WCD, providing a treatment or therapy shock to the patient requires significantly more power than merely providing monitoring of the patient's physiological signals. In such cases, while the WCD may continue to monitor the patient's ECG or other physiological signals with such a compromised battery, it may fail to provide adequate treatment shock(s) if the patient is experiencing, for example, ventricular fibrillation (VF), ventricular tachycardia (VT), or other life-threatening shockable arrhythmias. The systems and methods disclosed herein advantageously mitigate such risks by provide visual and/or other feedback to a user alerting them to the situation so that corrective action may be taken. As yet another example of a potential risk scenario, if a patient improperly and/or incorrectly inserts a charged battery into the WCD monitor/controller, the controller may not receive power and, as such, may not provide safety-critical monitoring of and treatment to the patient. Once again, the systems and methods disclosed herein advantageously mitigate such risks by provide visual and/or other feedback to a user alerting them to the situation.

To address these and other obstacles to successful execution of, and patient adherence to, proper battery maintenance tasks, including correct insertion, removal, verification that the battery is properly charging, and/or verification that the battery is properly providing power to the device, systems and processes for providing feedback regarding certain battery operations including battery insertion are described herein. For example, a rechargeable battery for a wearable medical device such as a WCD can include battery circuitry and a feedback mechanism configured to provide feedback indicating whether the rechargeable battery is properly inserted into either the wearable medical device or a battery charger. For example, the battery circuitry can be configured to determine whether the rechargeable battery is physically and properly inserted into the wearable medical device or battery charger such that the rechargeable battery is fully inserted into a receiving battery well and/or has established an electrical and/or mechanical connection with the wearable medical device or battery charger. In other examples, the battery circuitry can be configured to determine whether current is flowing out of the rechargeable battery (e.g., the rechargeable battery is inserted into and powering the wearable medical device) and/or whether current is flowing into the rechargeable battery (e.g., the rechargeable battery is inserted into the charger and is being charged). The feedback mechanisms and/or processes as described herein can be implemented by mechanical and/or electrical features that are operably coupled to the battery circuitry. For example, the feedback mechanism can include mechanical or electrical features that are configured to output a positive feedback if the battery circuitry provides an indication that the rechargeable battery is properly inserted into the wearable medical device or battery charger. Conversely, the feedback mechanism can be configured to output negative feedback if the battery circuitry provides an indication that the rechargeable battery is improperly inserted into the wearable medical device or battery charger. Positive or negative feedback can be provided via the feedback mechanism through human-perceptible indications that are visual, audible, and/or tactile in nature, in confirming that the rechargeable battery is either properly or improperly inserted into the wearable medical device or battery charger. Non-limiting examples of such positive and negative feedbacks are provided in greater detail below.

In some examples, the battery circuitry and the feedback mechanism can be integrated into the device configured to receive the rechargeable battery. For example, the battery circuitry and the feedback mechanism can be integrated directly into the wearable medical device and/or the battery charger. For example, as described herein, a WCD system can include one or more sensing electrodes for collecting electrical signals indicative of cardiac activity of a patient, one or more therapy electrodes configured to provide one or more treatment shocks to the patient as necessary, and a medical device controller. The controller can include a battery well disposed within its housing and configured to receive a rechargeable battery. The controller can further include battery circuitry configured to detect whether the battery is inserted into the battery well and provide human-perceptible confirmation that the battery has been inserted into the battery well and is providing power to the WCD.

In certain examples, a mechanical attachment mechanism is provided that is displaced or otherwise physically altered upon insertion of a rechargeable battery into a wearable medical device and/or battery charger. In such examples, the mechanical attachment mechanism can include or be operably coupled to a visual indicator that is configured to provide visual feedback to a patient inserting the rechargeable battery into a battery well as described herein. For example, the mechanical attachment mechanism and the visual indicator can be configured to operate in concert such that the visual indicator transitions from a first state (e.g., displaying a first predetermined color, such as the color red) when the battery is not properly inserted to a second state (e.g., displaying a second predetermined color, such as the color green) when the battery is properly inserted.

These examples, and various other similar examples of benefits and advantages of the techniques, processes, and approaches as provided herein, are described in additional detail below.

The various battery insertion detection and feedback devices and processes described herein are implemented, in some examples, with removable and rechargeable batteries configured to provide power to certain types of medical devices. For instance, some examples include a patient monitoring and treatment device. Patient monitoring and treatment devices are used to monitor and record various physiological and/or vital signals for a patient and provide treatment to a patient when necessary. For patients at risk of a cardiac arrhythmia, specialized cardiac monitoring and/or treatment devices such as a cardiac event monitoring device, a WCD, or a hospital wearable defibrillator can be prescribed to and worn by the patient for an extended period of time. For example, a patient having an elevated risk of sudden cardiac death, unexplained syncope, prior symptoms of heart failure, an ejection fraction of less than 45%, less than 35%, or other such threshold deemed of concern by a physician, and other similar patients in a state of degraded cardiac health can be prescribed a specialized cardiac monitoring and/or treatment device.

For example, a WCD such as the LifeVest® Wearable Cardioverter Defibrillator from ZOLL Medical Corporation (Chelmsford, MA), can be prescribed to the patient. As described in further detail below, such a device includes a garment that is configured to be worn about the torso of the patient. The garment can be configured to house various components such as ECG sensing electrodes and therapy electrodes. The components in the garment can be operably connected to a monitoring device that is configured to receive and process signals from the ECG sensing electrodes to determine a patient's cardiac condition and, if necessary, provide treatment to the patient using the therapy electrodes.

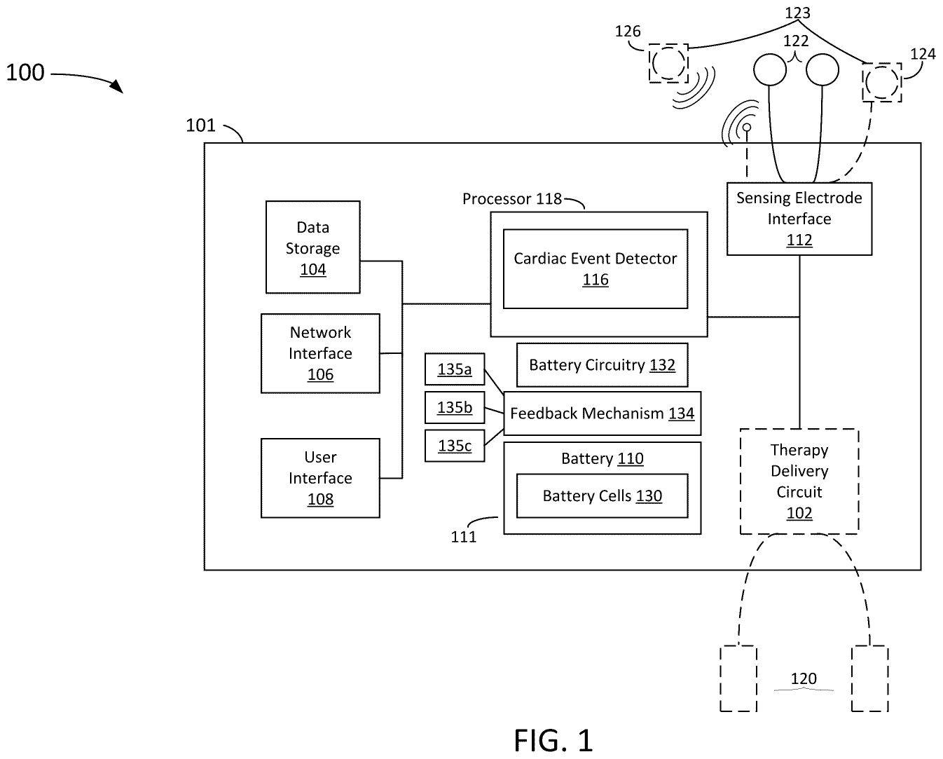

FIG. 1 illustrates an example component-level view of the medical device controller 100 included in, for example, a wearable medical device such as a WCD. As shown in FIG. 1, the medical device controller 100 can include a housing 101 configured to house a therapy delivery circuitry 102 configured to provide one or more therapeutic shocks to the patient via at least two therapy electrodes 120, a data storage 104, a network interface 106, a user interface 108, at least one rechargeable battery 110 (e.g., within a battery chamber configured for such purpose), a sensor interface 112 (e.g., to interface with both ECG sensing electrodes 122 and non-ECG physiological sensors 123 such as motion sensors, vibrational sensors, lung fluid sensors, infrared and near-infrared-based pulse oxygen sensor, blood pressure sensors, among others), a cardiac event detector 116, and least one processor 118.

In some examples, the patient monitoring medical device can include a medical device controller 100 that includes like components as those described above but does not include the therapy delivery circuitry 102 and the therapy electrodes 120 (shown in dotted lines). That is, in certain implementations, the medical device can include only ECG monitoring components and not provide therapy to the patient. In such implementations, the construction of the patient monitoring medical device is similar in many respects as a WCD medical device controller 100 but need not include the therapy delivery circuitry 102 and associated therapy electrodes 120.

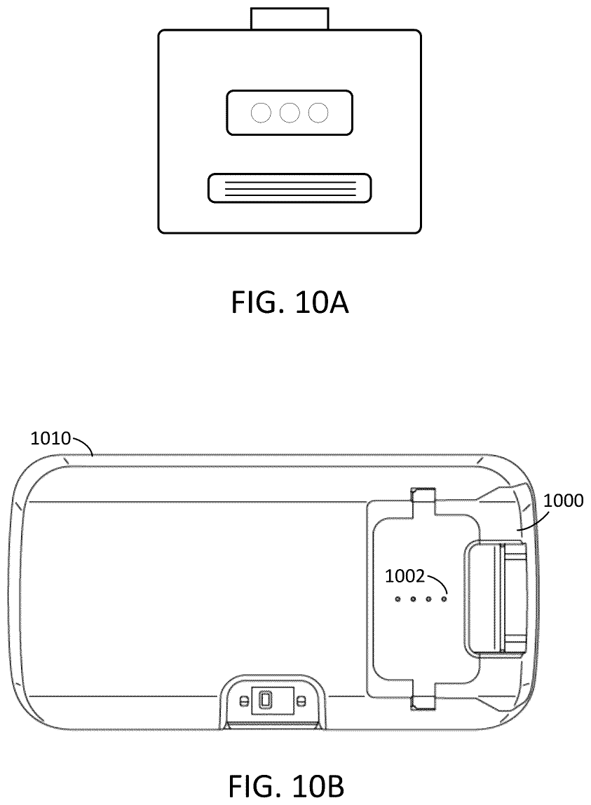

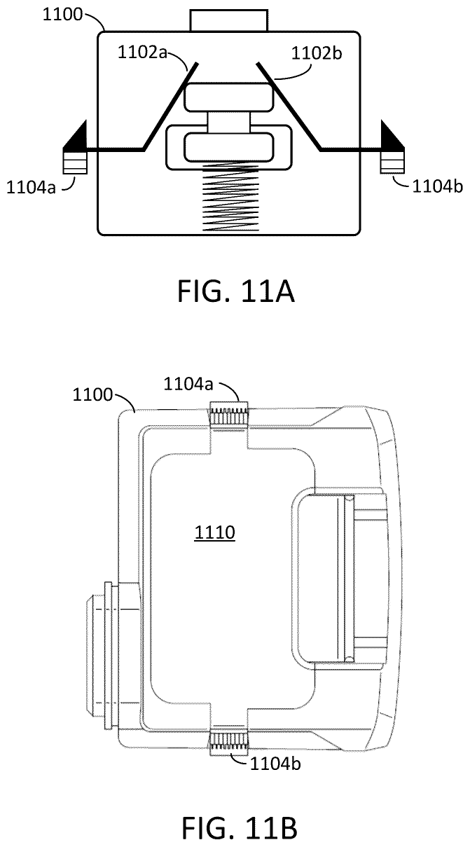

In certain implementations, the controller 100 can further include one or more components for determining whether the rechargeable battery 110 is properly inserted into the controller and providing power to one or more additional components within the controller. For example, as shown in FIG. 1, the controller 100 can include battery circuitry 132 configured to determine whether the rechargeable battery 110 is properly inserted into the controller 100. The controller 100 can also include at least one feedback mechanism 134 coupled to the battery circuitry 132 and configured to provide feedback to the user of the controller. Non-limiting examples of feedback mechanism 134 are described in further detail below in connection with FIGS. 10A, 10B, 11A and 11B, along with accompanying disclosure. For example, the feedback mechanism 134 can be configured to provide a visual, audio, and/or tactile feedback to the user of the controller 100 when the rechargeable battery 110 is properly inserted into the controller. For example, the feedback mechanism 134 can be configured to provide a visual feedback to a user through, for example, a display 135a or other similar output device configured to provide a visual indicator to a user of the controller 100. Similarly, the feedback mechanism 134 can be configured to provide an audio feedback to a user though, for example, a speaker 135b or other similar output device configured to provide an audio indicator to a user of the controller 100. In other examples, the feedback mechanism can be configured to provide a tactile feedback to a user through, for example, a tactile feedback device 135c including, for example, a vibration generating device or other similar device configured to provide a tactile indicator to a user of the controller 100. As an example, feedback mechanism 134 is an LED or LCD display. As another example, feedback mechanism 134 is an LED indicator. As an example, feedback mechanism 134 is a speaker for outputting predetermined human-perceptible audible alerts and/or voice messages. As an example, feedback mechanism 134 includes a vibration motor configured to issue a predetermined vibratory pattern (e.g., 0.5 seconds ON, 0.5 seconds OFF) or a signature pattern (e.g., 3 short vibrations followed by a long vibration).

Upon proper insertion, the rechargeable battery 110 is configured to provide power to the one or more therapy electrodes 120 to delivery one or more treatment shocks to the patient as needed. Similarly, upon proper insertion, the rechargeable battery is configured to provide power to the controller 100 to monitor electrical signals indicative of the cardiac activity of the patient for any cardiac arrhythmias. As such, by receiving feedback that the rechargeable battery 110 is properly inserted into the controller 100, the patient knows that the controller is properly monitoring the patient's cardiac activity and that the patient will be treated with one or more therapeutic shocks if needed.

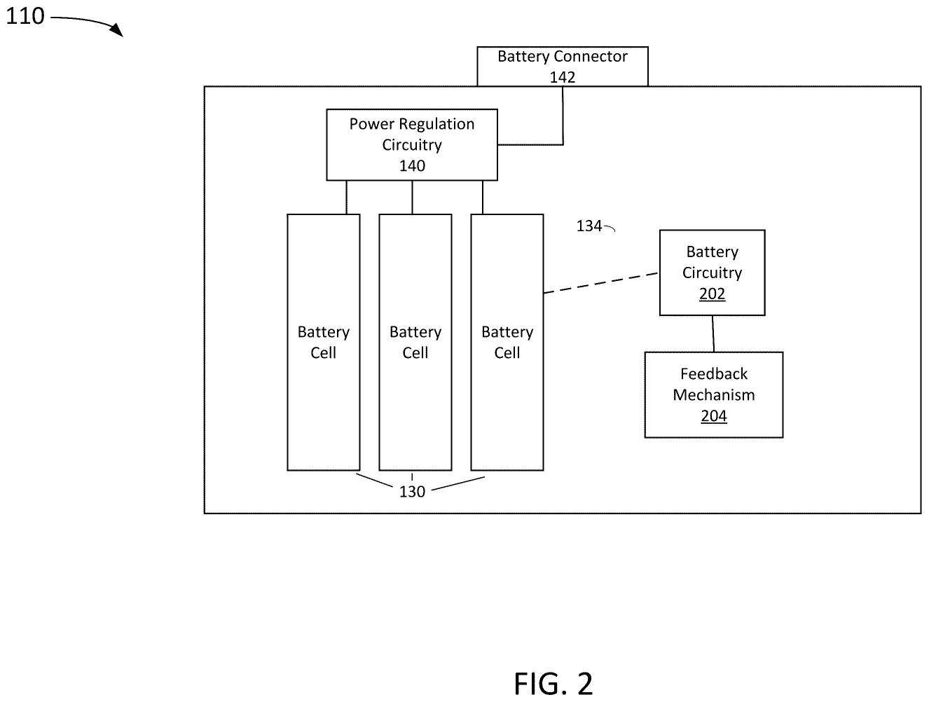

As further shown in FIG. 1, the housing 101 can include a battery well 111 or other similar receptacle for receiving the rechargeable battery 110 such that the rechargeable battery established an electrical connection with the controller 100. The rechargeable battery 110 can include various other components as described herein. For example, the rechargeable battery 110 can include one or more battery cells 130. In certain implementations, such as that as described in FIG. 2, the rechargeable battery 110 can include the battery circuitry 132 and the feedback mechanism 134 as described herein. The rechargeable battery 110 is described in additional detail in the following discussion of FIG. 2 through FIG. 4F. FIG. 1 is also described in greater detail below.

FIG. 2 illustrates a more detailed view of rechargeable battery 110. For example, as shown in FIG. 2, the rechargeable battery 110 can include a set of battery cells 130. As shown in FIG. 2, three battery cells 130 are provided by way of example only. Depending upon the design of the rechargeable battery 110, and the expected power output requirements of the rechargeable battery, the number of battery cells 130 can be varied. For example, the rechargeable battery 110 can include two battery cells 130, four battery cells, six battery cells, nine battery cells, and other similar quantities of battery cells.

As further shown in FIG. 2, the rechargeable battery 110 can further include power regulation circuitry 140. The power regulation circuitry 140 can be configured to condition and provide power to another device such as a wearable medical device as described herein via battery connector 142. Conversely, during charging, the power regulation circuitry 140 can be configured to receive power from the battery charger via the battery connector 142 and, if necessary, condition the power for charging the power cells 130.

As also shown in FIG. 2, the rechargeable battery 110 can include battery circuitry 202 configured to determine whether the rechargeable battery 110 is properly inserted into the controller 100 as shown, for example, in FIG. 1. The rechargeable battery 110 can also include at least one feedback mechanism 204 coupled to the battery circuitry 202 and configured to provide feedback to a user inserting, for example, the rechargeable battery into the controller 100. For example, the feedback mechanism 204 can be configured to provide a visual, audio, and/or tactile feedback to the user of the rechargeable battery 110 when the rechargeable battery is properly inserted into the controller 100. As an example, feedback mechanism 204 is an LED or LCD display. As another example, feedback mechanism 204 is an LED indicator. As an example, feedback mechanism 204 is a speaker for outputting predetermined human-perceptible audible alerts and/or voice messages. As an example, feedback mechanism 204 includes a vibration motor configured to issue a predetermined vibratory pattern (e.g., 0.5 seconds ON, 0.5 seconds OFF) or a signature pattern (e.g., 3 short vibrations followed by a long vibration). Other non-limiting examples of feedback mechanism 134 are described in further detail below in connection with FIGS. 10A, 10B, 11A and 11B, along with accompanying disclosure.

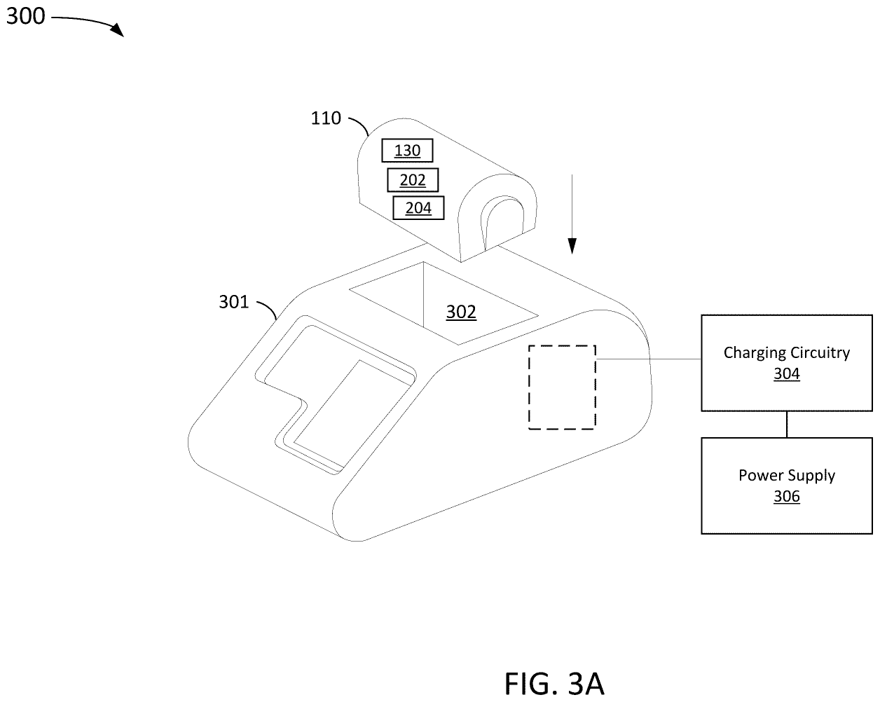

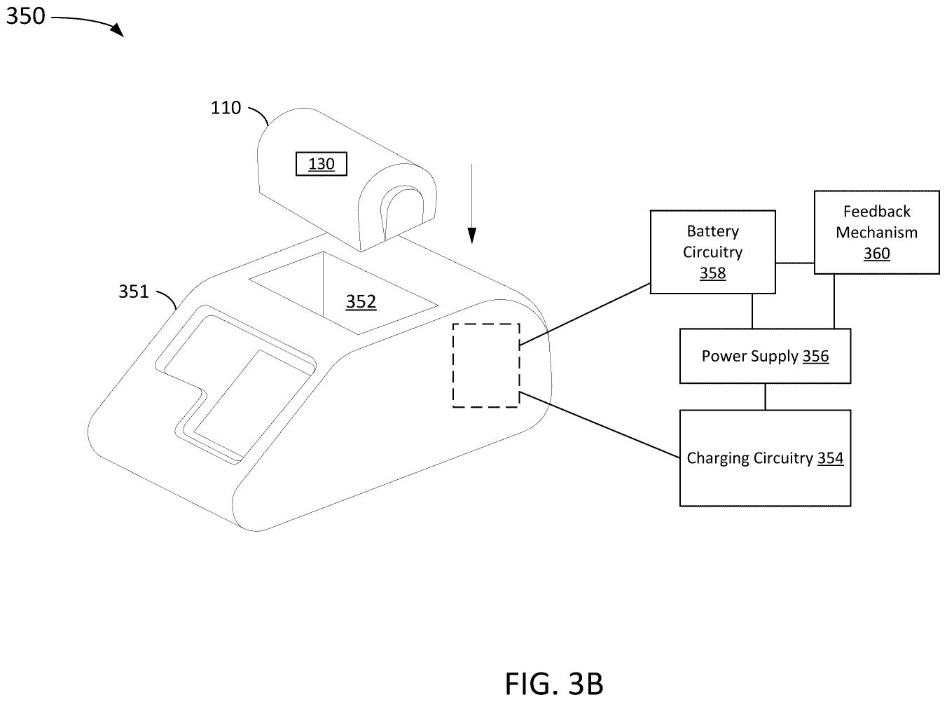

FIG. 3A illustrates an example component-level view of a battery charger 300 included with, for example, a wearable medical device such as a WCD when the medical device is prescribed to a patient. As shown in FIG. 3A, the battery charger 300 can include a housing 301 configured to house various components of the battery charger. For example, the housing 301 can be configured to include a battery receiving well 302 or other similar receptacle for receiving a rechargeable battery as described herein. The battery receiving well 302 can include a recessed or similarly shaped cavity configured to physically receive at least a portion of the rechargeable battery 110. The battery receiving well 302 can further include one or more electrical connectors configured to establish an electrical connection between the rechargeable battery 110 and charging circuitry 304. The charging circuitry 304 can include various electrical components arranged to condition power received from a power supply 306 into one or more electrical signals such as a charging current suitable for charging the rechargeable battery 110. For example, the power supply 306 can be a plug or other similar connector configured to plug into an electrical wall outlet and receive main power at, for example, 120 volts and 10 amps. The charging circuitry 304 can be configured to convert the main power to an electrical signal having an associated voltage and amperage suitable for charging the rechargeable battery 110. For example, the charging circuitry 304 can be configured to convert the main power to about 5 volts at about 2.5 amps.

As further shown in FIG. 3A, the rechargeable battery 110 includes the battery circuitry 202 and the feedback mechanism 204 as described herein. In such an example, the battery circuitry 202 and the feedback mechanism 204 can be configured to provide feedback to a user inserting the rechargeable battery 110 into the charger 300, thereby indicating when the rechargeable battery is properly inserted into the charger.

Additionally or alternatively, the battery circuitry and the feedback mechanism as described herein can be integrated into the battery charger rather than the rechargeable battery as described above in FIG. 3A. For example, FIG. 3B depicts another example battery charger 350. Battery charger 350 includes a housing 351 configured to house various components of the battery charger. For example, the housing 351 can be configured to include a battery receiving well 352 or other similar receptacle for receiving a rechargeable battery as described herein. Similar to the battery receiving well 302 as shown in FIG. 3A and described above, the battery receiving well 352 can include a recessed or similarly shaped cavity configured to physically receive at least a portion of the rechargeable battery 110. The battery receiving well 352 can further include one or more electrical connectors configured to establish an electrical connection between the rechargeable battery 110 and charging circuitry 354. The charging circuitry 354 can include various electrical components arranged to condition power received from a power supply 356 into one or more electrical signals such as a charging current suitable for charging the rechargeable battery 110 as described above.

As further shown in FIG. 3B, the charger 350 can include a battery circuitry 358 and a feedback mechanism 360 as described herein. In such an example, the battery circuitry 358 and the feedback mechanism 360 can be configured to provide feedback to a user inserting the rechargeable battery 110 into the charger 350, thereby indicating when the rechargeable battery is fully and properly inserted into the charger.

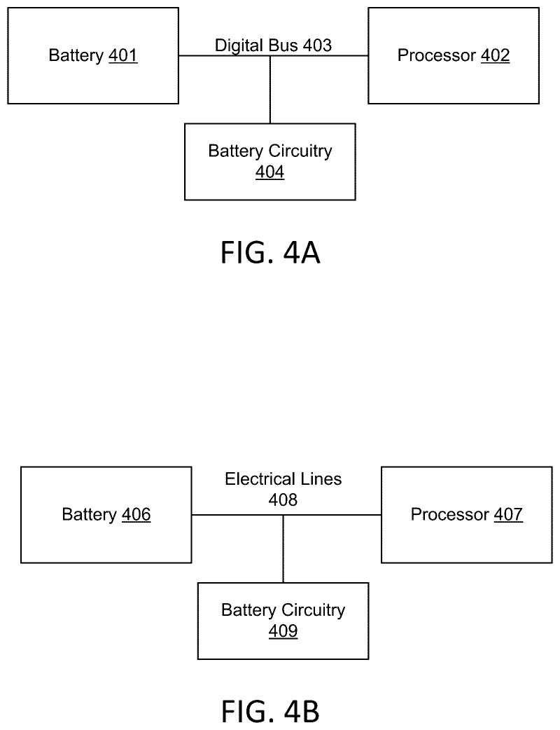

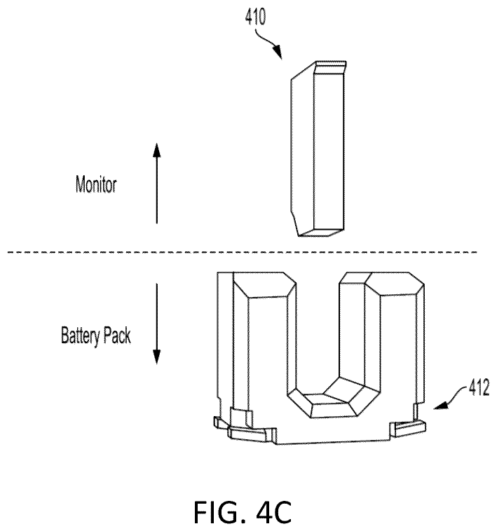

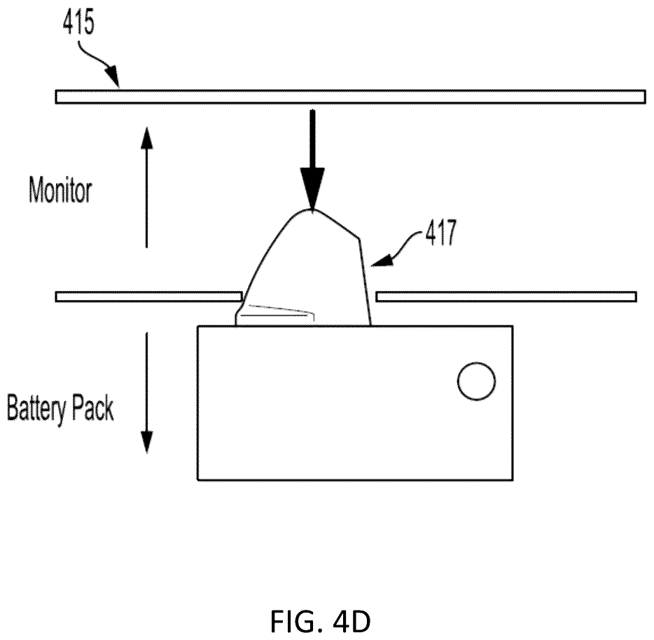

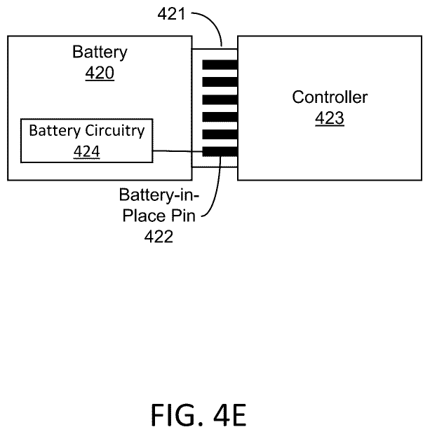

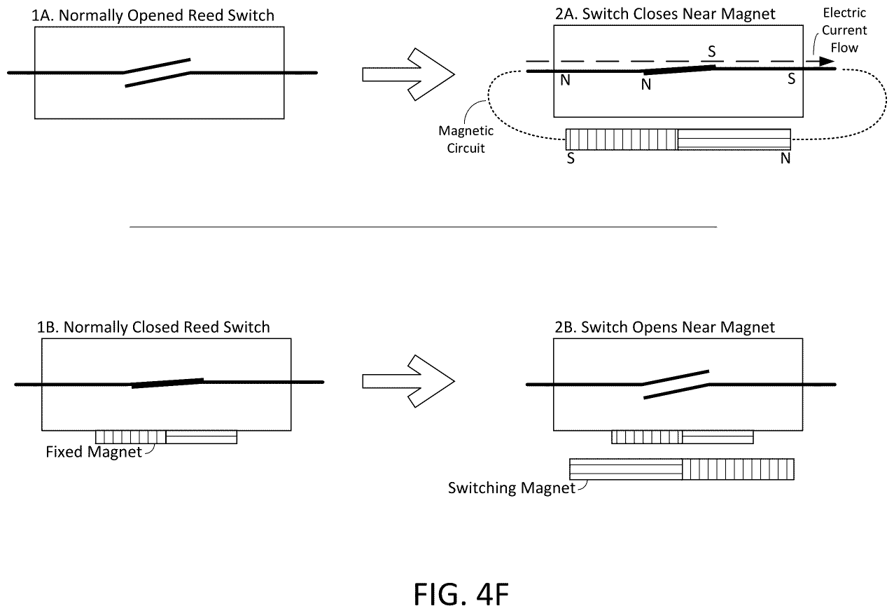

Depending upon the design of the rechargeable battery 110, the medical device controller 100, and the battery chargers 300 and 350, the battery circuitry (e.g., battery circuitry 132, 202, and 358 as shown in FIGS. 1-3B) as described herein can be designed and implemented in various manners. For example, Table 1 as shown below outlines design types for implementing the battery circuitries (e.g., battery circuitry 202 of FIG. 3A, or battery circuitry 358 of FIG. 3B) as well as a brief function summary and a commercial example, each type being described in greater detail below with reference to one or more of FIGS. 4A-4F. As examples, feedback mechanism 204 of FIG. 3A and/or feedback mechanism 360 of FIG. 3B are an LED or LCD display. As another example, feedback mechanism 134 is an LED indicator. As an example, feedback mechanism 204 of FIG. 3A and/or feedback mechanism 360 of FIG. 3B are a speaker for outputting predetermined human-perceptible audible alerts and/or voice messages. As an example, feedback mechanism 204 of FIG. 3A and/or feedback mechanism 360 of FIG. 3B include a vibration motor configured to issue a predetermined vibratory pattern (e.g., 0.5 seconds ON, 0.5 seconds OFF) or a signature pattern (e.g., 3 short vibrations followed by a long vibration). Other non-limiting examples of feedback mechanism 134 are described in further detail below in connection with FIGS. 10A, 10B, 11A, and 11B, along with accompanying disclosure.

TABLE 1

| Example battery circuitry | |
|---|---|
| (e.g., battery circuitry | |
| 202 of FIG. 3A, or | |
| battery circuitry 358 of | |
| FIG. 3B) | Example function |
| Digital I/O Monitoring | Monitor connection bus between battery and monitor/controller for |
| | data transfer |
| Analog I/O Monitoring | Measure for active discharge of battery cells or current flow from |
| | battery cells |
| Hall Effect Sensor | Measure magnetic field generated by a portion of the |
| | monitor/controller to determine if battery is inserted into |
| | monitor/controller |
| | Example device: Texas Instruments DRV5053 |
| Optical Sensor | Use imaging such as infrared-based proximity detection to |
| | determine if battery is inserted into monitor/controller |
| | Example devices: Sharp GP2YOD805ZOF, Everlight EAITRAA1 |
| | (transmissive) |
| Ultrasonic Sensor | Use ultrasonic sound detection to determine if battery is inserted |
| | into monitor/controller |
| | Example device: Baumer UNCK 09G8914/IO |
| Proximity Switch | Physical interface that detects physical presence of the |
| | monitor/controller as a result of displacement of at least a portion of |
| | the switch |
| | Example device: Panasonic ESE22 Detector Switches |
| Battery Latch | Physical interface that is manually or automatically displaced when |
| | the battery is inserted into the monitor/controller and may remain at |
| | least partially displaced when properly inserted |
| | Example device: Crouzet M30 CPM723015C |
| Dedicated Electrical | A dedicated pin on the Battery/Monitor electrical connector |
| Connection Monitor | |
| Magnetic reed switch | Switch changes condition/position when battery is inserted |

For example, as listed in Table 1, the battery circuitry can include digital input/output (I/O) monitoring. The digital I/O monitoring can include monitoring a connection such as an electrical bus between the medical device controller and the rechargeable battery, or the battery charger and the rechargeable battery, to determine if the rechargeable battery is properly inserted and electrically coupled to either the medical device controller or the battery charger.

For example, as shown in FIG. 4A, a rechargeable battery 401 can be electrically coupled to a processor 402 of a wearable medical device controller via a digital bus 403. The battery circuitry 404 can monitor one or more lines in the electrical bus 403 for activity indicative of a connection between the rechargeable battery 401 and the processor 402. For example, the electrical bus 403 can include a status line that is high when the rechargeable battery 401 is connected to the processor 402 and, conversely, is low when there is no connection. The battery circuitry 404 can be configured to monitor the status line condition to determine whether an electrical connection is established between the rechargeable battery 401 and the processor 402.

In another example as listed in Table 1, the battery circuitry can include monitoring for analog signals between the medical device controller and the rechargeable battery, or the battery charger and the rechargeable battery, to determine if the rechargeable battery is properly inserted and electrically coupled to either the medical device controller or the battery charger.

For example, as shown in FIG. 4B, a rechargeable battery 406 can be electrically coupled to a processor 407 of a wearable medical device controller via one or more electrical lines 408. A battery circuitry 409 can monitor one or more lines in the electrical lines 408 for activity indicative of a connection between the rechargeable battery 406 and the processor 407. For example, the battery circuitry 409 can be configured to monitor the electrical lines 408 to monitor for current flow from the rechargeable battery 406 to the processor 407, thereby providing an indication that the rechargeable battery and the processor are electrically coupled.

In addition to monitoring circuitry configured to monitor for a digital or analog signal that can be indicative of a connection between a rechargeable battery and a wearable medical device controller or battery charger, the battery circuitry can further include one or more physical sensors and/or detection mechanisms for determining if a rechargeable battery is properly inserted into another device.

In another example as shown in Table 1, the battery circuitry can include a Hall Effect sensor that is configured to measure a magnetic field generated by, for example, a magnet that is positioned adjacent to a position where the rechargeable battery is inserted into the wearable medical device controller or battery charger. For example, a magnet can be integrated into the housing 301 of the battery charger near the battery receiving well 302 as described above. Upon insertion of the rechargeable battery into the battery receiving well 202, the Hall Effect sensor included in the battery circuitry as described herein can measure the magnetic field as generated by the magnet, thereby providing an indication that the rechargeable battery is properly inserted into the battery charger or the wearable medical device controller.

Other powered proximity-type sensors are also included in Table 1. For example, the battery circuitry can include an optical sensor such as an infrared proximity detector that is configured to measure a distance between a portion of the rechargeable battery and a portion of the wearable medical device controller and/or battery charger. Another proximity-type sensor as shown in Table 1 includes an ultrasonic sensor that is configured to emit an ultrasonic sound and measure reflected sound waves to determine whether a rechargeable battery is properly inserted into a wearable medical device controller and/or a battery charger.

FIG. 4C illustrates an example of various powered proximity sensors such as the Hall Effect sensor, optical sensor, and ultrasonic sensor as described above. For example, the wearable medical device controller can include a detectable feature 410 such as a protruding piece of the housing of the controller. The rechargeable battery can also include a sensor 412 such as a Hall Effect sensor, an optical sensor, and an ultrasonic sensor as described herein and listed in Table 1. If the sensor 412 is a Hall Effect sensor, the detectable feature 410 of the medical device controller can include a magnet. If the sensor 412 is an optical sensor, the detectable feature 410 of the medical device controller can include one or more reflective surfaces configured to reflect any light emitted by the optical sensor. If the sensor 412 is an ultrasonic sensor, the detectable feature 410 can be made of a sonically reflective material that is shaped to reflect sound emitted by the ultrasonic sensor back at the sensor.

In addition to powered proximity-type sensors, the battery circuitry can include mechanical proximity-type sensors as well. For example, the battery circuitry can include a proximity switch that is at least partially depressed or otherwise displaced when the rechargeable battery is inserted into, for example, a wearable medical device controller. In another example, another mechanical proximity interface can include a battery latch that is depressed by the patient when inserting the rechargeable battery. In certain implementations, at least a portion of the battery latch can remain displaced when the rechargeable battery is properly inserted into the wearable medical device controller. In both examples, the battery circuitry can include a switch that is directly connected to the proximity switch or battery latch, and the status of the switch (e.g., opened or closed) can provide a direct indication of whether the rechargeable battery is properly inserted into the wearable medical device controller.

For example, FIG. 4D illustrates a sample mechanical proximity switch. As the rechargeable battery is inserted into the wearable medical device controller, a portion 415 of the controller can be positioned to depress a proximity switch 417 on the rechargeable battery. While the rechargeable battery remains properly inserted in the controller, the proximity switch 417 remains at least partially depressed as described above.

Another battery circuitry type as noted in Table 1 can include a dedicated electrical connection monitor. For example, the monitor can be configured to detect a signal on a dedicated pin on the connector between the rechargeable battery and the wearable medical device controller. In certain implementations, the dedicated pin can provide a loop-back function that provides a signal to the rechargeable battery that there is an electrical connection with the controller. When implemented, the dedicated electrical connection monitor can be configured to confirm via measurement that an electrical connection has been made between the rechargeable battery and the wearable medical device controller.

For example, as shown in FIG. 4E, a rechargeable battery 420 can be electrically connected to a controller 423 via a battery connector 421. The battery connector 421 can include at least one battery-in-place pin 422 that is monitored by battery circuitry 424. As noted above, the battery circuitry 424 can monitor for any changes in the output of the battery-in-place pin and, upon detecting a change, provide an indication to the alarm circuitry as described herein.

Another battery circuitry example can be the use of a mechanical switch such as a reed switch that reacts to the presence of, for example, a magnet. Rather than use a powered sensor such as Hall Effect sensor, a reed switch provides for a mechanical solution. For example, as shown in FIG. 4F, a reed switch can be normally open or normally closed. When in proximity of a magnet, the normally open reed switch moves to a closed state and, conversely, the normally closed reed switch moves to an open state. Such a switch can be integrated into the battery circuitry as described herein to provide an additional type of mechanical detachment detection.

It should be noted that, in the above examples, when determining a connection between a rechargeable battery and a wearable medical device controller is discussed, the same concepts and techniques can be used to determine a connection between the rechargeable battery and a battery charger. Conversely, in the above examples, when determining a connection between a rechargeable battery and a battery charger is discussed, the same concepts and techniques can be used to determine a connection between the rechargeable battery and a wearable medical device controller.

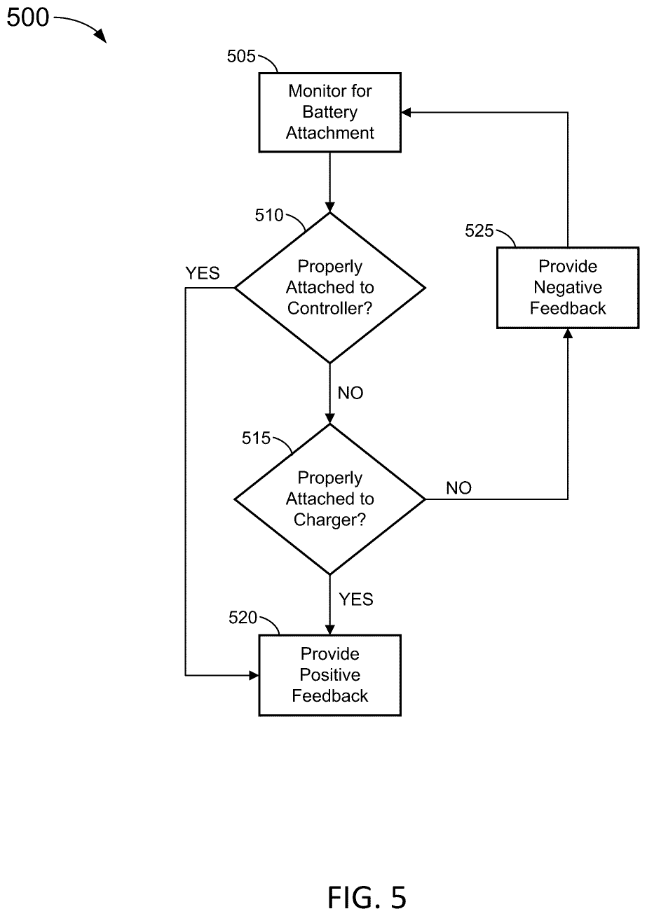

FIG. 5 illustrates a sample process 500 for providing feedback regarding proper insertion of a rechargeable battery into, for example, a medical device controller or a battery charger as described herein. In certain implementations, the battery circuitry as described herein can include or be operably coupled to a processor (e.g., processor 118 as described above) or other similar computing device that is configured to perform a set of instructions to perform a process such as process 500 as described herein. For example, the processor can monitor 505 for battery attachment. For example, the processor can be operably coupled to one or more of the battery circuit types as listed in Table 1 and described above.

In certain implementations, the processor can monitor one or more connections between the rechargeable battery and the wearable medical device for the presence or status of one or more signals that can be indicative of a connection between the rechargeable battery and the wearable medical device controller. Examples of such signals indicative of a connection can include a digital communication signal (e.g., as outlined in FIG. 4A and described above), an analog communication signal (e.g., as outlined in FIG. 4B and described above), an electrical connection signal associated with, for example, one or more connection pins of the rechargeable battery (e.g., as shown in FIG. 4E and described above), and a location signal indicating a physical location of the rechargeable battery (e.g., as shown in FIGS. 4C, 4D, and 4F and described above).

As further shown in FIG. 5, the output of the battery circuit can be monitored 505 and one or more determinations can be made. For example, the processor can determine 510 whether the rechargeable battery is properly attached to the wearable medical device controller. If the processor does determine 510 that the rechargeable battery is properly attached to the controller, the processor can provide 520 positive feedback regarding insertion of the rechargeable battery into the medical device controller. If the processor does not determine 510 that the rechargeable battery is properly attached to the controller, the processor can determine 515 if the rechargeable battery is attached to the battery charger. If the processor determines 515 that the battery is attached to the battery charger, the processor can provide 520 positive feedback regarding insertion of the rechargeable battery into the battery charger. Conversely, if the processor does not determine 515 that the rechargeable battery is properly inserted into the battery charger, the processor can provide 525 negative feedback and continue to monitor 505 the output of the battery circuitry for battery attachment.

It should be noted that the process 500 as shown in FIG. 5 is shown by way of example only. In actual implementation, the process 500 can include fewer or additional steps, various steps can be combined and/or reordered, and other similar adjustments to the process can be made. For example, in implementation, the monitoring 505 for battery attachment as well as the determining 510 whether the rechargeable battery is attached to the controller and the determining 515 whether the rechargeable battery is attached to the battery charger can be combined into a single monitoring and determinization step.

In a use-case example, the above described processes and techniques can be implemented to reduce the risk that a patient wearing a wearable medical device forgets or improperly inserts a rechargeable battery into the device when replacing a depleted rechargeable battery with a charged rechargeable battery during a battery swap. The above described processes and techniques can also reduce the risk that the patient will not properly insert the depleted rechargeable battery into the battery charger for charging. For example, an elderly heart failure patient may be prescribed a WCD for continuous wear while also being given a battery charger and two rechargeable batteries. Upon the initial fitting of the wearable medical device, the patient can be instructed that the rechargeable batteries should be swapped every 24 hours and that, upon removal from the controller, the depleted rechargeable battery should be placed on the battery charger and left until the next battery swap. During the first battery swap, the patient may be confused or forget the instructions and fail to properly insert the depleted rechargeable battery onto the battery charger. Using the techniques and processes as described herein, the patient can quickly determine whether they have properly inserted the rechargeable battery into the battery charger. Similarly, when inserting a recently charged battery into the medical device controller, the patient can receive similar feedback that the rechargeable battery is properly inserted into the medical device controller and that the controller is receiving power from the properly inserted battery.

In addition to including battery circuitry configured to determine when the battery is properly inserted into a battery well as described above, additional mechanical feedback and electromechanical detection and feedback techniques can be used to determine when a rechargeable battery is properly inserted into a battery well in, for example, a medical device controller and/or a battery charger. For example, one or both of the rechargeable battery and the receiving battery well can include a mechanical attachment configured to detect the insertion of the rechargeable battery into an interior volume of the battery well. In certain implementations, the mechanical attachment can protrude from an exterior surface of the rechargeable battery. Similarly, the mechanical attachment can be disposed within an interior volume of the receiving battery well.

In certain implementations, the mechanical attachment can be configured to be disposed, depressed, rotated, or otherwise moved in response to the rechargeable battery being inserted into the battery well. The mechanical attachment can be configured to work in concert with a visual indicator. In some examples, the visual indicator can be integrated into the mechanical attachment. In other examples, the visual indicator can be operably coupled to the mechanical attachment and configured to provide feedback regarding the insertion of the rechargeable battery into the battery well. As the rechargeable battery is inserted, the visual indicator can transition from a first state to a second state (e.g., from red to green). Once the rechargeable battery is fully and properly inserted into the battery well such that the battery is inserted into the receiving battery well and an electrical connection is established between the battery and another device, the visual indicator can remain in the second state, thereby providing visual feedback that the rechargeable battery is properly inserted.

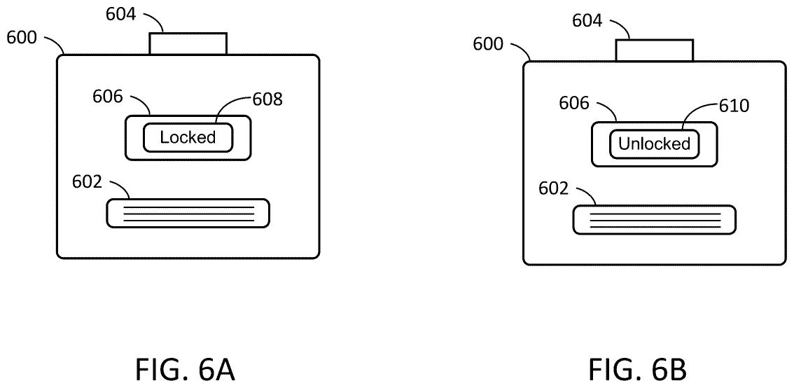

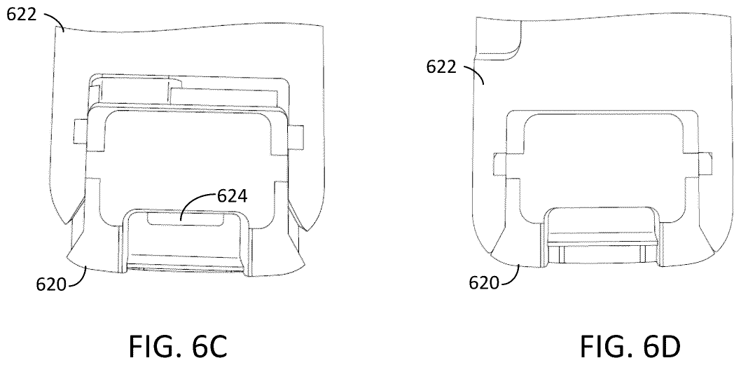

FIGS. 6A-14 provide additional examples of mechanical attachments, visual indicators, and other similar battery insertion features that can be used to provide feedback to a patient when a rechargeable battery as described herein is properly inserted into a battery receiving well. It should be noted that examples of mechanical attachment components, feedback mechanisms, and other related components as shown in FIGS. 6A-14 and described below can be integrated into devices and systems configured to include battery circuitry and feedback mechanisms as shown in FIGS. 1-5 and described above to provide more robust proper battery insertion detection and related patient feedback.

FIGS. 6A and 6B illustrate examples of a rechargeable battery including a visual indicator configured to provide patient feedback regarding whether the rechargeable battery is properly inserted into a receiving battery well. For example, as shown in FIG. 6A, a rechargeable battery 600 can include a release mechanism 602, a battery connector 604, and a visual indicator 606. As shown in FIG. 6A, the visual indicator 606 is configured to output display 608 including the word “locked” when the rechargeable battery 600 is properly inserted into a receiving battery well.

In certain implementations, upon manipulation of the release mechanism 602, the rechargeable battery 600 may become disengaged or otherwise be improperly positioned within the battery well. In such an example, the displayed output as shown by the visual indicator 606 can be updated. For example, as shown in FIG. 6B, the visual indicator 606 is updated to output display 610 including the word “unlocked.” In such an example, the visual indicator 606 can provide an indication to the patient that the battery 600 is not properly inserted into a receiving battery well.

It should be noted that visual indicators 606 is shown as displaying words indicating the proper or improper insertion of the rechargeable battery 600 by way of example only in FIGS. 6A and 6B. In actual implementation, the visual indicator 606 can be configured to display, for example, one or more colors indicating the current state of the rechargeable battery 600 and whether it is properly locked within a receiving well, a pattern or other set of symbols indicating the current state of the rechargeable battery and whether it is properly locked within a receiving well, and other similar visual indicators.

FIGS. 6C and 6D illustrate wireframe drawings of a sample rechargeable battery 620 being inserted into, for example, a battery well of a wearable medical device 622. As shown in FIG. 6C, when initially being inserted into the battery well, the rechargeable battery 620 can have a visual indicator 624 that is visible to the patient and/or the person inserting the battery into the wearable medical device 622. As shown in FIG. 6D, once the rechargeable battery 620 is fully inserted into the battery well of the wearable medical device 622, the visual indicator can be covered, updated, or otherwise changes as described herein to indicate that the rechargeable battery is fully inserted.

In certain implementations as described herein, a rechargeable battery could include a mechanical attachment and/or set of mechanical attachment features that are configured to lock the battery within a receiving battery well. In some examples, the mechanical attachment features can be configured to physically adjust the visual indicator as the battery is inserted into the receiving battery well. For example, the rechargeable battery can include a set of movable pawls that are configured to be displaced as the battery is inserted into the receiving battery well. As the pawls are displaced, the visual indicator can also be displaced or otherwise moves such that the output of the visual indicator is updated to reflect the current status of the rechargeable battery and provide an indication as to whether the rechargeable battery is properly inserted into the receiving battery well.

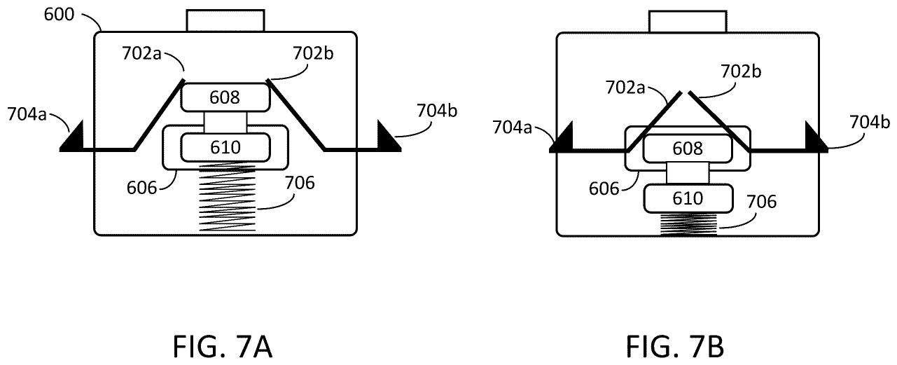

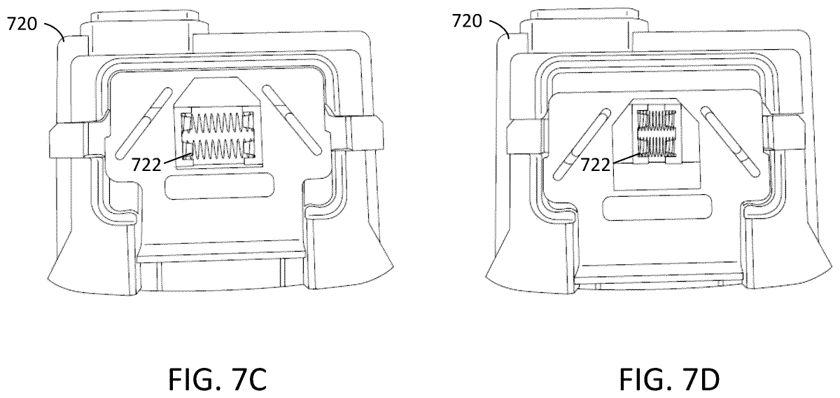

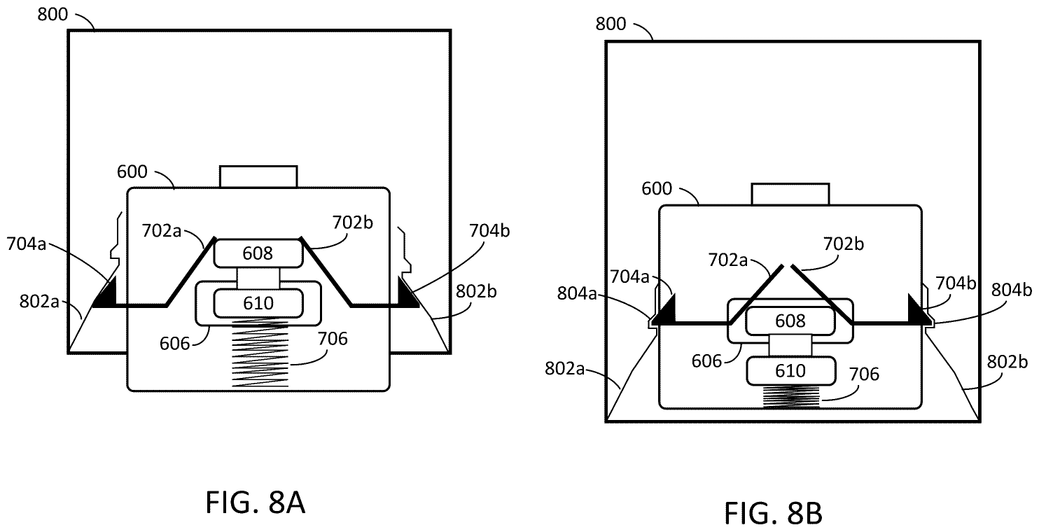

For example, FIGS. 7A-8B illustrate an example rechargeable battery including a mechanical attachment feature that is configured to physically adjust a visual indicator as the battery is inserted into a receiving battery well. In the example as illustrated in FIGS. 7A-8B, rechargeable battery 600 is shown from the reverse side as shown in FIGS. 6A and 6B, i.e., the front facing side in FIGS. 7A-8B is the face that would be inserted and face into a receiving battery well.

As shown in FIG. 7A, the rechargeable battery 600 can include one or more mechanical attachment features that are configured to be displaced or otherwise moved when the battery is inserted into a receiving battery well. In this example, the mechanical attachment features include movable pawls 702a and 702b. As shown in FIG. 7A, the movable pawls 702a and 702b can be shaped such that at least a portion 704a and 704b of the movable pawls extends beyond the housing of the rechargeable battery 600. Additionally, as shown in FIG. 7A, each of the movable pawls 702a and 702b can be positioned such that it contacts at least a portion of the output displays 608 and 610 (shown physically coupled in FIG. 7A by way of example only). As the movable pawls 702a and 702b are displaced inward by, for example, inserting the rechargeable battery 600 into a battery receiving well, the position of the displays 608 and 610 can be altered, thereby changing what is shown in the visual indicator 606 as described above. As further shown in FIG. 7A, a spring 706 or other similar device can be positioned adjacent to the displays 608 and 610 and configured to exert an opposition force on the displays to return the displays to their original position when the movable pawls 702a and 702b return to their original position.

FIG. 7B illustrates an example of the rechargeable battery 600 when the movable pawls 702a and 702b are displaced inwards as a result, for example, of the battery being inserted into a receiving battery well. As shown in FIG. 7B, the inward movement of the movable pawls 702a and 702b has moved display 608 into the visual indicator and moved display 610 out of the visual indicator, also resulting in the compression of spring 706. In such an example, when the rechargeable battery 600 is removed from the receiving battery well, the spring 706 can exert an opposition force on the displays 610 and 608 to return them to their original position as shown in FIG. 7A. Such a movement can also cause the movable pawls 702a and 702b to return to their original position as shown in FIG. 7A.

FIGS. 7C and 7D illustrate wireframe drawings of a sample rechargeable battery 720 having movable pawls such as those shown in FIGS. 7A and 7B and described above. As shown in FIGS. 7C and 7D, the springs 722 can be repositioned and oriented to oppose the force as applied by, for example, the pawls. As such, it should be noted that the position and orientation of the spring 706 and springs 722 as shown in FIGS. 7A-7D are provided by way of example only.

FIGS. 8A and 8B correspond to FIGS. 7A and 7B respectively. However, both FIGS. 8A and 8B include a receiving battery well 800 for receiving rechargeable battery 600. As shown in FIG. 8A, as the rechargeable battery is initially inserted into receiving battery well 800, the movable pawls 702a and 702b are extended, display 610 is visible in the visual indicator 606, and the spring 706 is extended. In this example, as the rechargeable battery is inserted into the receiving battery well 800, the portions 704a and 704b contacts a corresponding receiving portion 802a and 802b molded or otherwise formed within the receiving battery well. As shown in FIG. 8A, the receiving portions 802a and 802b are shaped such that, upon further insertion of the rechargeable battery 600 into the receiving battery well 800, the receiving portions contact the portions 704a and 704b of the movable pawls 702a and 702b and cause the movable pawls to be displaced in an inward direction toward the center of the battery. As described above, such movement of the movable pawls 702a and 702b can result in compression of spring 706 and movement of the displays 608 and 610 within the visual indicator 606.

As shown in FIG. 8B, the rechargeable battery 600 is fully and properly inserted into the receiving battery well 800. In such an example, the shape and position of the receiving portions 802a and 802b have caused additional displacement of the movable pawls 702a and 702b respectively. The displacement of the movable pawls 702a and 702b has caused movement of the displays 608 and 610 and compression of the spring 706. Additionally, when the rechargeable battery 600 is properly inserted into the battery receiving well 800, the portions 704a and 704b of the movable pawls 702a and 702b can be seated, inserted, or otherwise receiving within a receiving feature. For example, as shown in FIG. 8B, a receiving detent 804a can be configured to receive portion 704a of the movable pawl 702a when the rechargeable battery 600 is fully inserted into the receiving battery well 800. Similarly, a receiving detent 804b can be configured to receive portion 704b of the movable pawl 702b when the rechargeable battery 600 is fully inserted into the receiving battery well 800. The receiving detents 804a and 804b can also act to oppose accidental release of the rechargeable battery 600 from the receiving battery well 800 until the battery is released by the patient upon activation of, for example, the battery release mechanism 602 as shown in FIGS. 6A and 6B as described above. For example, upon activation of the battery release mechanism 602, the movable pawls 70a and 70b can further be displaced inward such that the portions 704a and 704b can be removed or otherwise be unseated from the receiving detents 804a and 804b, thereby allowing for removable of the rechargeable battery 600 from the receiving battery well 800.

It should be noted that the mechanical attachment features as shown in FIGS. 7A-8B are provided by way of example only. In actual implementation, various features of the design as shown in FIGS. 7A-8B can be altered. For example, the geometry of the movable pawls 702a and 702b can be adjusted based upon the overall shape of the rechargeable battery 600 and the position of the visual indicator 606. Similarly, the geometry and position of the receiving portions 802a and 802b can be adjusted to correspond to the shape and position of the movable pawls 702a and 702b respectively. Additionally, the inclusion of the spring 706 is provided by way of example only. Additional force exertion components can be used to counteract the movement of the mechanical attachment features and adjust the visual indicator display upon removal of the rechargeable battery from the receiving battery well as described herein.

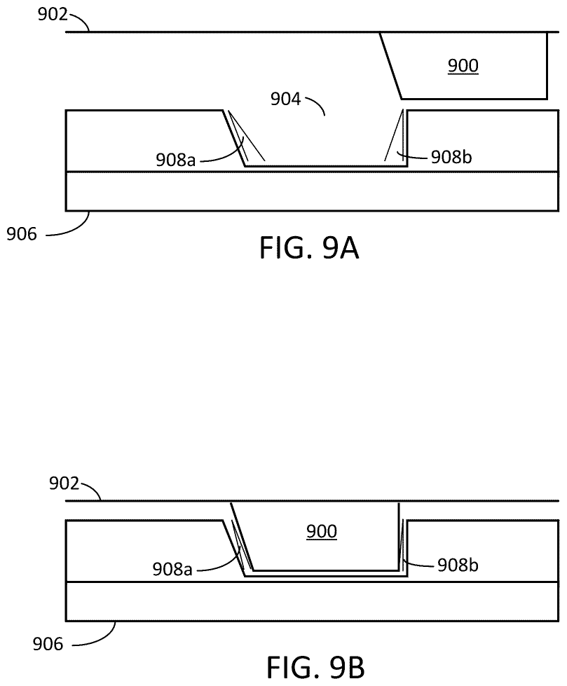

In some examples, a rechargeable battery and receiving battery well can be configured to provide an indication of when the rechargeable battery is partially inserted into the battery well. For example, FIGS. 9A and 9B provide an overview of an example where the receiving well includes one or more mechanical components configured to provide an indication or feedback regarding position of the rechargeable battery within the receiving battery well. As shown in FIG. 9A, a pawl 900 can extend from the side of a rechargeable battery 902. As the rechargeable battery 902 is inserted into a receiving battery well 906, the pawl can align with receiving portion 904. As further shown in FIG. 9A, the receiving portion 904 can include one or more additional insertion detection components. For example, the receiving portion 904 can include springs 908a and 908b that are configured to be displaced upon insertion of the pawl 900 into the receiving portion or aperture. In certain implementations, the displacement of only one of the springs 908a or 908b can indicate that the pawl 900 is only partially inserted into the receiving portion 904. In such an example, each of the springs 908a and 908b can be connected to a visual indicator as described herein. The visual indicator can be configured to provide various notifications indicating if the pawl 900 is partially inserted into the receiving portion 904 (e.g., only one of the springs 908a or 908b is displaced) or if the pawl is fully and properly inserted into the receiving portion (e.g., both springs are displaced). As shown in FIG. 9B, the pawl 900 is properly and fully inserted within the receiving portion 904 and both the springs 908a and 908b are displaced.

In addition to mechanical attachment features and related visual indicators, additional attachment and indicator components or types can be included. For example, as shown in FIG. 10A, a rechargeable battery 1000 can include a visual indicator 1002 that includes one or more LEDs. In some examples, upon insertion of the rechargeable battery 1000 into a receiving battery well, the visual indicator 1002 can display one or more patterns and/or colors of light indicating whether the battery is properly inserted. For example, if the visual indicator 1002 includes all red LEDs, the rechargeable battery 1000 may be completely removed from or improperly inserted into the receiving battery well. Similarly, if the visual indicator 1002 includes a mixture of red and green LEDs, the rechargeable battery 1000 may be partially inserted into the battery receiving well (e.g., as described above in the discussion of FIGS. 9A and 9B). If the visual indicator 1002 includes all green LEDs, the rechargeable battery 1000 may be fully and properly inserted into the receiving battery well such that the battery is seated within the receiving battery well and an electrical connection is stabled between the battery and an electrical connector within the battery well.

FIG. 10B illustrates an alternative example of the rechargeable battery 1000 being inserted into, for example, a wearable medical device 1010. In this example, the visual indicators 1002 are arranged such that they are visible when the battery 1000 is inserted into the wearable medical device 1004.

It should be noted that visual indicator 1002 is described above as including colored LEDs by way of example only. In certain implementations, the visual indicator 1002 can be configured to output dynamically updated text or patterns that provide an indication of the status of the rechargeable battery 1000 with regard to its insertion into a receiving battery well. Additionally, as described herein, additional feedback mechanisms such as an audio output feedback device and a tactile feedback device can be included. For example, the audio output feedback device can be adjustable when the patient is initially fitted with a wearable medical device. A caregiver can administer a hearing test to the patient and record what audio frequencies the patient responds to and/or hears clearly. Based upon the test, the output of the audio output feedback device can be updated accordingly. Similarly, a tactile feedback device can be configured to provide a tactile sensation such as vibrating at a particular frequency that the patient responds to during fitting.

As described above, battery circuitry can be used to determine whether a battery is fully and properly inserted into a receiving battery well. As noted in FIGS. 4A-4F and the accompanying text above, various types of battery circuitry can be used. In certain implementations, at least a portion of the battery circuitry can be included in the rechargeable battery. For example, as shown in FIG. 11A, a rechargeable battery 1100 can include one or more electrical connectors configured to establish an electrical connection with at least a portion of a receiving battery well when the battery is properly inserted into the battery well. As shown in FIG. 11A, a movable pawl 1102a can include an electrical contact 1104a. Similarly, a movable pawl 1102b can include an electrical contact 1104b. When the rechargeable battery 1100 is properly inserted into a receiving battery well, each of the electrical contacts 1104a and 1104b can establish an electrical contact with a portion of the receiving battery well, thereby providing an indication that the battery is properly inserted.

FIG. 11B illustrates a similar example of the rechargeable battery 1100 as shown in FIG. 11A. In this example, the electrical contacts 1104a and 1104b can be seen protruding from housing 1110 of the rechargeable battery 1100. Similar to above, once the rechargeable battery 1100 is inserted into a receiving battery well, each of the electrical contacts 1104a and 1104b can establish an electrical contact with a portion of the receiving battery well, thereby providing an indication that the battery is properly inserted.

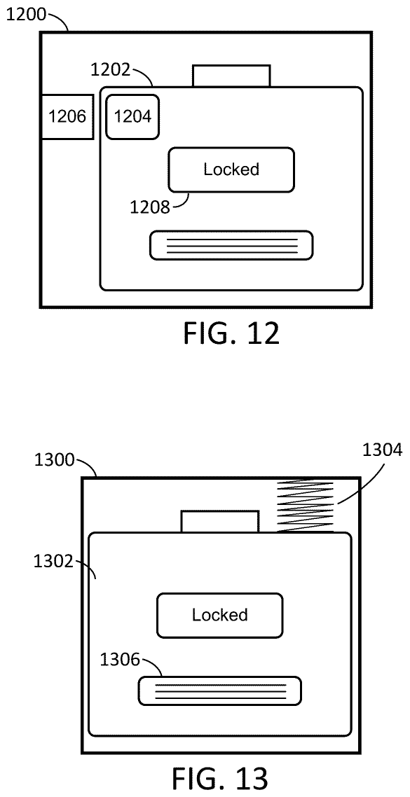

Similarly, as shown in FIG. 12, a rechargeable battery 1202 can include a sensor 1204 that is configured to detect the position of the battery within a receiving battery well 1200. For example, the sensor 1204 can be implemented as a Hall Effect sensor. As the rechargeable battery 1202 is inserted into the receiving battery well 1200, the Hall Effect sensor 1204 can detect a magnet 1206 integrated into a portion of the interior volume of the receiving battery well. Upon detection of the magnet, the Hall Effect sensor can output a signal that results in updating of a visual indicator 1208 on the rechargeable battery 1202.

It should be noted that the arrangement of components as shown in FIG. 12 can be altered based upon the design of the rechargeable battery and the receiving battery well. For example, the Hall Effect sensor can be integrated into the receiving battery well and a magnet can be integrated into the rechargeable battery. Similarly, a Hall Effect sensor and magnet is provided by way of example only. Additional sensors, such as those listed above in Table 1, can be used. For example, an optical sensor, an ultrasonic sensor, a proximity switch, a battery latch, and other similar sensors and circuitry can be used in a similar arrangement as shown in FIG. 12.

In certain implementations, when properly inserted, the rechargeable battery can latch properly in place within a receiving battery well. As shown in FIGS. 8A and 8B and described above, when properly inserted, a movable pawl or other similar mechanism can latch into a receiving portion of the receiving battery well. In some examples, the battery well can further include a spring or other similar force exerting component that is configured to oppose insertion of the rechargeable battery into the receiving battery well. For example, as shown in FIG. 13, as a rechargeable battery 1302 is inserted into a receiving battery well 1300, a spring 1304 can be configured to oppose insertion of the battery. Upon locking of the rechargeable battery 1302 into proper position, the spring 1304 can remain compressed and continue to exert an opposition force upon the battery. When the rechargeable battery 1302 is to be removed (e.g., upon activation of the release mechanism 1306, thereby causing one or more latching members to disengage with one or more receiving portions within the receiving battery well 1300), the spring 1304 can exert the opposition force onto the battery, thereby facilitating removal of the battery from the receiving battery well. The spring 1304 can also act as a safety feature. In an example where the rechargeable battery 1302 is not fully and properly inserted and latched within the receiving battery well 1300, the spring 1304 can at least partially eject the battery from the receiving battery well.

It should be noted that the spring 1304 is shown in FIG. 13 by way of example only. In some examples, a seal or other similarly compressible material can be included on either the rechargeable battery and/or the receiving battery well. For example, the battery seal is disposed between the battery to controller interface as disclosed in further detail below. The battery seal is, in examples, configured to meet an IP67 rating. In some embodiments, the battery seal has a liquid ingress protection rating of at least one of IPX3, IPX4, IPX5, IPX6, IPX7, or IPX8 as specified in international standard EN 60529 (British BS EN 60529:1992, European IEC 60509:1989). These ingress protection ratings are shown in Table 2.

TABLE 2

| Rating | Brief Description | Definition |
|---|---|---|
| IPX3 | Protected against | Water sprayed at an angle up to 60° on either side of |
| | spraying water | the vertical shall have no harmful effects. |
| IPX4 | Protected against | Water splashed against the enclosure from any |
| | splashing water | direction shall have no harmful effects |
| IPX5 | Protected against | Water projected in jets against the enclosure from any |
| | water jets | direction shall have no harmful effects |
| IPX6 | Protected against | Water projected in powerful jets against the enclosure |
| | powerful water jets | from any direction shall have no harmful effects |
| IPX7 | Protected against the | Ingress of water in quantities causing harmful effects |
| | effects of temporary | shall not be possible when the enclosure is |
| | immersion in water | temporarily immersed in water under standardized |
| | | conditions of pressure and time |
| IPX8 | Protected against the | Ingress of water in quantities causing harmful effects |
| | effects of continuous | shall not be possible when the enclosure is |
| | immersion in water | continuously immersed in water under conditions |
| | | which shall be agreed between manufacturer and user |
| | | but which are more severe than for numeral 7 |

In some embodiments, the battery seal has solid particle ingress protection rating of one of IP3X, IP4X, IP5X, or IP6X as specified in international standard EN 60529 (British BS EN 60529:1992, European IEC 60509:1989). These ingress protection ratings are shown in Table 3.

TABLE 3

| Rating | Brief Description | Definition |
|---|---|---|
| IPX3 | Protected against | The object probe, sphere |
| | solid foreign | of 2.5 mm diameter shall |
| | objects of 2.5 mm | not penetrate at all |
| | diameter and | |
| | greater | |
| IPX4 | Protected against | The object probe, sphere |
| | solid foreign | of 1.0 mm diameter shall |
| | objects of 1.0 | not penetrate at all |
| | mm diameter | |
| | and greater | |
| IPX5 | Dust-protected | Ingress of dust is not totally |
| | | prevented, but dust shall not |
| | | penetrate in a quantity to |
| | | interfere with satisfactory |
| | | operation of the apparatus |
| | | or to impair safety |
| IPX6 | Dust-tight | No ingress of dust. |

In some embodiments, the battery seal includes liquid ingress protection rating of at least one of IPX3, IPX4, IPX5, IPX6, IPX7, or IPX8 as specified in international standard EN 60529 (British BS EN 60529:1992, European IEC 60509:1989) and a solid particle ingress protection rating of one of IP3X, IP4X, IP5X, or IP6X as specified in international standard EN 60529 (British BS EN 60529: 1992, European IEC 60509: 1989).

Upon proper insertion of the rechargeable battery, the seal can compress to both provide an opposition force as well as to provide protection to, for example, one or more electrical connections between the rechargeable battery and the medical device controller that result from the rechargeable battery being inserted into the receiving battery well.

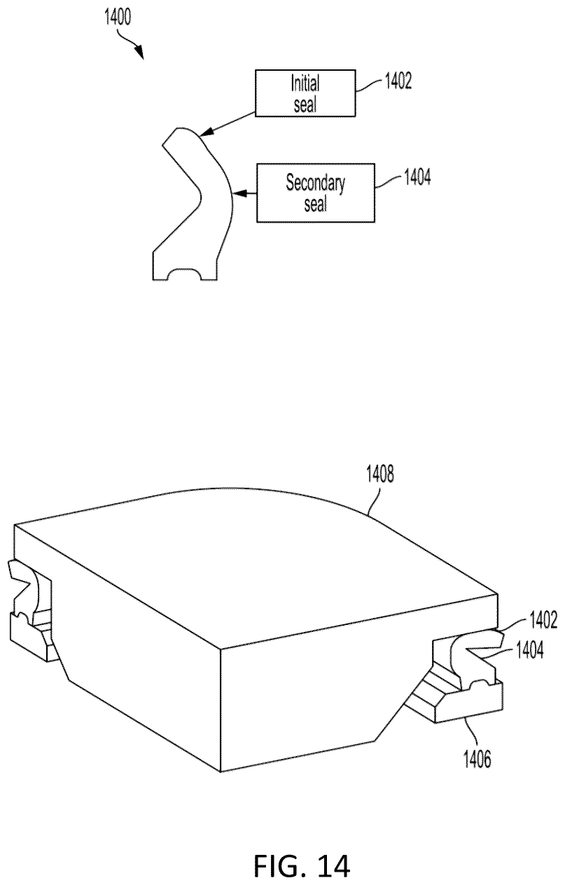

For example, as shown in FIG. 14, a seal 1400 can be included on the battery and positioned about the electrical connector of the battery. The seal 1400 can be configured such that, upon compression, the seal forms an initial seal 1402 against an initial receiving surface. Upon further compression, the seal 1400 can be further configured to form a secondary seal 1404 against a secondary surface as a result of further compression of the seal. As further shown in FIG. 14, the seal 1400 can be integrated into a mating surface 1406 of a rechargeable battery, the mating surface being positioned about an electrical connector of the rechargeable battery. Upon inserting into a receiving battery well, the seal can contact a receiving portion 1408. As the seal 1400 initially contacts the receiving portion 1408, the seal compresses to form the initial seal 1402 against the receiving surface. As the seal 1400 is further compressed, the seal forms the secondary seal 1404 against the receiving portion 1408. Once the rechargeable battery is latched within the receiving battery well, the seal 1400 can remain in a compressed state and continue to exert an opposition force between the mating surface 1406 of the rechargeable battery and the receiving portion 1408 of the receiving battery well. Upon release of the rechargeable battery latch from the receiving well portion, the seal 1400 can return to its original shape, thereby causing at least a partial ejection of the rechargeable battery from the receiving battery well. Similarly, in an example where the rechargeable battery is not fully and properly inserted and latched within the receiving battery well, the seal 1400 can at least partially eject the battery from the receiving battery well, thereby providing an indication that the rechargeable battery is not properly inserted.

In some implementations, as described herein, the battery removal is achieved via a use of a plurality (e.g., at least two) retention pawls with a sliding release lever. In some implementations, as described herein, the battery latch indication includes a predetermined color indicator, e.g., red color latched indicator when the battery is not fully inserted. In some implementations, as described herein, the battery can be located towards an end on a rear side of the controller (e.g., on side opposite the side on which the user interface is disposed). For example, the device status can be provided via one or more indicator lights (e.g., LED-based lights) disposed on a housing of the controller. For example, the indicator can be a tri-color indicator (e.g., includes predetermined colors such as green, yellow, and red). The indicator lights can be used to depict the following states of the device based on battery insertion status. For example, the indicator light can indicate “Ready for use” status by displaying a green slow breath frequency (e.g., a 0.1 to 0.3 Hz), a “Medium priority alert” status by displaying a yellow pulse frequency (e.g., 0.5 Hz), and a “High priority alert” status by displaying red flash frequency (e.g., 2 Hz).

Table 4 below provides a summary of battery-related features in conjunction with an example wearable cardiac monitoring/treatment device as disclosed herein.

TABLE 4

| Feature | Description |
|---|---|
| Battery removal | Two latching retention pawls with one sliding release lever. |
| Latch Activation | One handed latch release |
| Battery Latch Indication | Red latched indicator when not fully inserted |
| Battery location | Located on rear of monitor at the end of the monitor. |
| Battery Safety | Discharge FET, UV protection with delay |
| Battery Safety | Active Cell Balancing Circuit |
| Battery Safety | Thermal gap pads for accurate individual cell temperature |
| | measurements |
| Battery Safety | HW Fuse |
| Battery Safety | Built in pack over voltage protection (OVP) |
| System Communications | Pack EEPROM provides data transfer from Charger to |
| | Battery to Controller to Network |
| Temperature Monitoring | Individual cell voltages available over SMBUS interface |
| Power Contacts | Redundant ground and power contacts |
| Charging Characteristics | C/3 Charge Rate |
| Charging Characteristics | Thermal Suspend |
| Pack Construction | Cells assembled with and retained by a core holder |
| Pack Construction | Rigid-flex protection board for improved assembly |
| Size | 2.88 in × 3.06 in × 1.26 in |
| Weight | 225 g |
| Environmental | 60 C. discharge temp |
| State of Health | Coulomb Counting Impedance Tracking Gas Gauge |
| Drop Performance | Drop performance (18×) |
| Case Assembly | Ultrasonic Welding on cases |
| Sealing | IP67 sealing for battery |

Table 5 below provides a summary of battery-related features in conjunction with an example wearable cardiac monitoring/treatment device as disclosed herein.

TABLE 5

| Feature | Description |
|---|---|
| LED Indicators | 4 LED indicators |
| Latch features | Latching Insertion |
| Pack Insertion | Insertion consistent with Monitor for reduction of |
| Method | patient training needs. |
| Assembly | Modular circuit design |
| Architecture | |
| Self Test | Charge circuit in-system-test load |
| Pack Insertion | Battery detection by interrogating predetermined |
| Detection | data/clock line |
| Alarm System | Speaker-Plays complex alarm sounds |
| Ease of Removal | Anti-skid features for one-handed side operation |
| Spill Mitigation | Splash proof connector protection, Spill mitigation |
| Test Accessibility | External test port |
| Sealing on Battery | IP54 sealing on battery to charger interface |
| Interface | |
| Modularity | Serviceable/Replaceable DC jack assembly |
| Pack Wakeup | Charging dependent on gauge comms |
| Thermal Protection | Onboard temperature sensing of critical circuitry |
| | (thermistors) |
| Drop Performance | 1 meter 18× drop performance |
| Pressure Mitigation | Pressure equalization patch |
| Sealing | IP54 sealing |

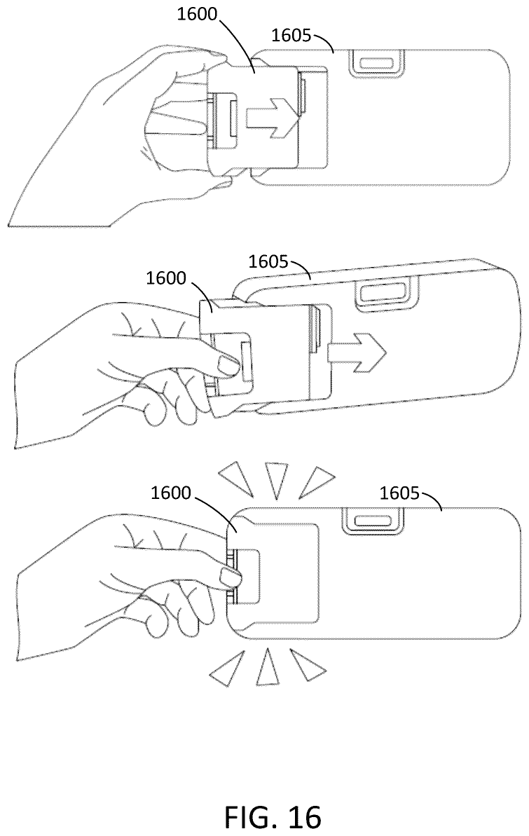

An example study, conducted according to a predetermined protocol described below, focused on collecting biomechanical forces and participant preferences during simulated battery insertion and removal activities relating to the systems, methods, and devices described herein. For this study, the battery systems, devices, and techniques shown in at least FIG. 16 was used. For examples, as shown in FIG. 16, a patient participant was instructed to insert a battery 1600 into a medical device 1605. As shown in FIG. 16, insertion of the battery included, for example, applying an insertion force upon the battery 1600 to insert the battery into the medical device 1605. A reverse process as that shown in FIG. 16 was used to collect force information for removal of the battery 1600 from the medical device 1605 for the participants as well.

In the study, twenty-five participants representative of the wearable cardioverter defibrillator user population were asked to simulate battery insertion and removal, pushing or pulling on the battery and battery latch while exerting as much force as possible. Forces were measured using force gauges installed into two mock monitor-battery test fixtures—one for insertion and one for removal. Participants executed 12 different activities (six insertion tasks and six removal tasks), designated as “standard” tasks. Each task was repeated three times. Participants were also asked to perform insertion and removal using their preferred methods as two additional tasks, bringing the total tasks to 14.

Per statistical analysis, body and arm position did not influence the participants' ability to exert their maximum compressive (insertion) or tensile (removal) force on the battery. The minimum force exerted during a standard insertion task was 5.4 lbf—the participant was seated with their arms raised off the table while using their forefingers to apply maximum force.

There was a statistically significant difference in maximum removal forces exerted by participants, with forces exerted during forefinger removal being significantly higher than those exerted during thumb removal. However, the minimum force exerted during a standard removal task was 5.2 lbf while the participant was standing with their arms raised and using their forefingers to apply maximum removal force.

At the conclusion of the activities, participants were asked to insert and remove the battery from a prototype WCD controller in a way that felt most natural to them. Their preferred methods and subjective feedback were recorded. Seventeen of 25 participants used their palm to insert the battery whilst inserting it into the monitor. Eleven of these participants commented that inserting the battery came naturally to them and felt easy to insert while using their palm. The maximum exerted force generated when performing this preferred method of insertion was shown to be significantly greater than the force applied using forefingers in the same position.

In further detail, the evaluation of the devices, systems, and techniques described herein was conducted according to the following protocol. Twenty-five (25) participants were recruited for the study, and each attended a single study session to assess their hand strength while simulating battery insertion and removal tasks. Each session lasted approximately 10-15 minutes, and 14 tasks were performed in total (Table 6). Tasks were performed using the participant's dominant hand. At the start of the session, the participants received an overview of the required insertion and removal. techniques and were taught the proper form for each technique. In addition, participants were taught to accurately read the force values from each measurement device.

As tasks were conducted, the participant reported the recorded maximum force value to the moderator, who then visually confirmed the value by checking the device before zeroing (Zeroing the device between each trial ensured that the reference point from which all measurements were made was maintained at 0 lbf throughout the study and that no drift in the values occurred). Participants were instructed to apply as much force as possible using each technique and hold for approximately 2 seconds, as counted by the moderator. Each task was performed three times for a total of 42 trials. The participants were given time to rest as needed between trials to allow their arm to relax and minimize fatigue. In addition, the order of the tasks was rotated between each participant to reduce hand fatigue.

TABLE 6

| Task | | |
|---|---|---|
| Code | Task name | Task description |
| FG1AX | Latch Force | Participants will use their dominant hand's forefingers (including |
| | Insertion, | their index, middle, and ring fingers) to push the battery into the |
| | Forefinger | monitor while sitting. They will hold the monitor completely off the |
| | Sitting, Raised | table, so no part of the battery, monitor, or participant's arms are |
| | | contacting the table. Then, the participant will exert maximal force |
| | | while pushing. |
| FG1AR | Latch Force | Participants will use their dominant hand's forefingers (including |
| | Removal, | their index, middle, and ring fingers) to pull on the latch while |
| | Forefinger | sitting. They will hold the monitor completely off the table, so no |
| | Sitting, Raised | part of the battery, monitor, or participant's arms are contacting the |
| | | table. Then, the participant will exert maximal force while pulling. |
| FG1BX | Latch Force | Participants will use their dominant hand's forefingers (including |
| | Insertion, | their index, middle, and ring fingers) to push the battery into the |
| | Forefinger | monitor while sitting. They will rest the monitor and any |
| | Sitting, Resting | combination of their elbows, forearms, and hands on the table. |
| | | Then, the participant will exert maximal force while pushing. |
| FG1BR | Latch Force | Participants will use their dominant hand's forefingers (including |
| | Removal, | their index, middle, and ring fingers) to pull on the latch while |
| | Forefinger | sitting. They will rest the monitor and any combination of their |
| | Sitting, Resting | elbows, forearms, and hands on the table. Then, the participant will |
| | | exert maximal force while pulling. |
| FG2AX | Latch Force | Participants will use their dominant hand's thumb to push the |
| | Insertion, Thumb | battery into the monitor while sitting. They will hold the monitor |
| | Sitting, Raised | completely off the table, so no part of the battery, monitor, or |
| | | participant's arms are contacting the table. Then, the participant |
| | | will exert maximal force while pushing. |
| FG2AR | Latch Force | Participants will use their dominant hand's thumb to pull on the |
| | Removal, | latch while sitting. They will hold the monitor completely off the |
| | Thumb Sitting, | table, so no part of the battery, monitor, or participant's arms are |
| | Raised | contacting the table. Then, the participant will exert maximal force |
| | | while pulling. |
| FG2BX | Latch Force | Participants will use their dominant hand's thumb to push the |
| | Insertion, Thumb | battery into the monitor while sitting. They will rest the monitor and |
| | Sitting, Resting | any combination of their elbows, forearms, and hands on the table. |
| | | Then, the participant will exert maximal force while pushing. |
| FG2BR | Latch Force | Participants will use their dominant hand's thumb to pull on the |
| | Removal, | latch while sitting. They will rest the monitor and any combination |
| | Thumb Sitting, | of their elbows, forearms, and hands on the table. Then, the |
| | Resting | participant will exert maximal force while pulling. |
| FG3AX | Latch Force | Participants will use their dominant hand's forefingers (including |
| | Insertion, | their index, middle, and ring fingers) to push the battery into the |
| | Forefinger | monitor while standing. They will hold the monitor completely off |
| | Standing, | the table, so no part of the battery, monitor, or participant's arms |
| | Raised | are contacting the table. Then, the participant will exert maximal |
| | | force while pushing. |
| FG3AR | Latch Force | Participants will use their dominant hand's forefingers (including |
| | Removal, | their index, middle, and ring fingers) to pull on the latch while |
| | Forefinger | standing. They will hold the monitor completely off the table, so no |
| | Standing, | part of the battery, monitor, or participant's arms are contacting the |
| | Raised | table. Then, the participant will exert maximal force while pulling. |
| FG4AX | Latch Force | Participants will use their dominant hand's thumb to push the |
| | Insertion, Thumb | battery into the monitor while standing. They will hold the monitor |
| | Standing, | completely off the table, so no part of the battery, monitor, or |
| | Raised | participant's arms are contacting the table. Then, the participant |
| | | will exert maximal force while pushing. |
| FG4AR | Latch Force | Participants will use their dominant hand's thumb to pull on the |
| | Removal, | latch while standing. They will hold the monitor completely off the |
| | Thumb | table, so no part of the battery, monitor, or participant's arms are |
| | Standing, | contacting the table. Then, the participant will exert maximal force |
| | Raised | while pulling. |
| PAX | Preferred | Participants will be asked to insert the battery into a prototype |
| | Insertion | monitor using their preferred technique. This method may or may |
| | Technique | not be one of those discussed in a previous task. If the participant |
| | | uses a method not previously covered in earlier tasks, the |
| | | participant will be asked to repeat the method on a test fixture |
| | | using maximal force and will be asked why they prefer this |
| | | technique. |
| PAR | Preferred | Participants will be asked to remove the battery from a prototype |
| | Removal | monitor using their preferred technique. This method may or may |
| | Technique | not be one of those discussed in a previous task. If the participant |
| | | uses a method not previously covered in earlier tasks, the |
| | | participant will be asked to repeat the method on a test fixture |
| | | using maximal force and will be asked why they prefer this |
| | | technique. |

A summary of the recruited participants can be found in Table 7 below.

TABLE 7

| | |
|---|---|
| Age | 20-29 years of age: n = 5 |
| Groups | 30-39 years of age: n = 11 |
| | 40-49 years of age: n = 2 |
| | 50-59 years of age: n = 6 |
| | 60-69 years of age: n = 1 |
| Gender | Male: n = 20 |
| | Female: n = 5 |
| Dominant | Right: n = 25 |
| Hand | Left: n = 0 |
| Physical | Normal: 23 |
| Dexterity | Some: 2 |
| | Injured: 1 * |

* One participant injured their dominant wrist one day before the study but did not report any physical dexterity issues

The study took place in a private conference room setting in Pittsburgh, Pennsylvania. The study environmental conditions were designed to not impact the participants' experience or ability to perform the desired tasks. Two (2) test fixtures were used for data collection. Test fixtures consisted of a WCD monitor implementing the present methods, systems, and devices (the LifeVest WCD from ZOLL, Chelmsford) outer shell with a digital force gauge installed. One fixture was configured to record compressive force during insertion tasks. The other fixture was used for recording tensile force during removal tasks. A Mark-10 Series 2 Digital Force Gauge, Model M2-100 was installed inside each test fixture. One was used to measure tensile forces during battery removal, and the other was used to measure compressive forces during battery insertion.

Every participant completed each task three times. The average of these three data points was then calculated for each participant for every task they completed. The tasks were separated into two primary categories: Insertion and Removal.

The results of the study are as follows. Regarding battery insertion, the activities were conducted in six different body and arm positions, all using the participant's dominant hand. While seated, participants exerted maximum insertion force using their:
Forefingers, arms raised above the table (Activity FG1AX),
Forefingers, arms resting on the table (Activity FG1BX),
Thumb, arms raised above the table (Activity FG2AX),
Thumb, arms resting on the table (Activity FG2BX).

While standing, participants performed two additional insertion tasks using their forefingers and thumb as described below:
Forefingers, arms raised in front of them (Activity FG3AX), and
Thumb, arms raised in front of them (Activity FG4AX).

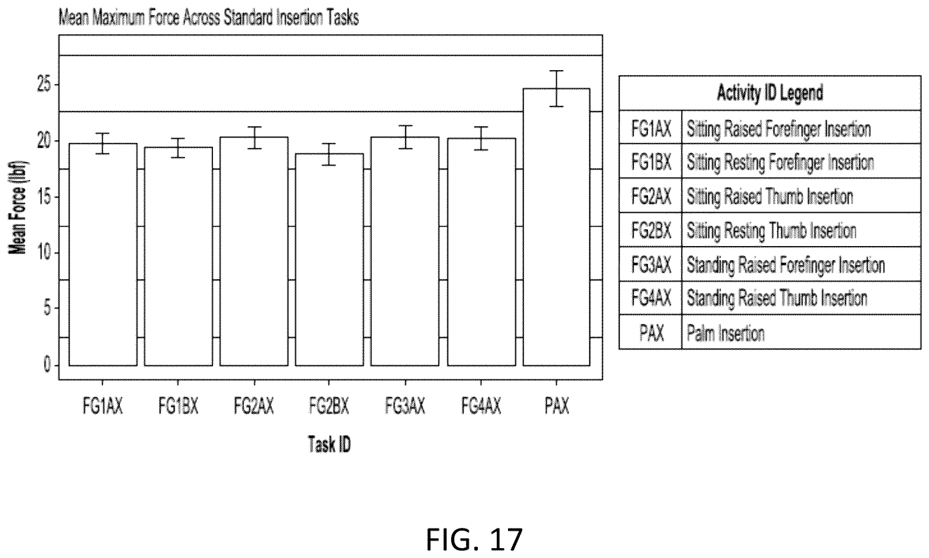

Participants performed an additional insertion activity at the end of the session, citing their preferred insertion technique. The mean maximum forced generated by participants for each insertion activity can be seen in FIG. 17 (the error bars depict statistical standard error). Further, the insertion force exerted by participants who preferred using their palm to apply force and insert the battery into the monitor are recorded here as task PAX (n=17). Further, when participants could choose their preferred insertion method, 17 of 25 participants chose to insert the battery using their palm. In two of those 17 instances, participants used a combination of the palm and forefingers. In one instance, the palm was used in combination with the thumb. Another eight participants used some part of their forefingers as their preferred insertion method.

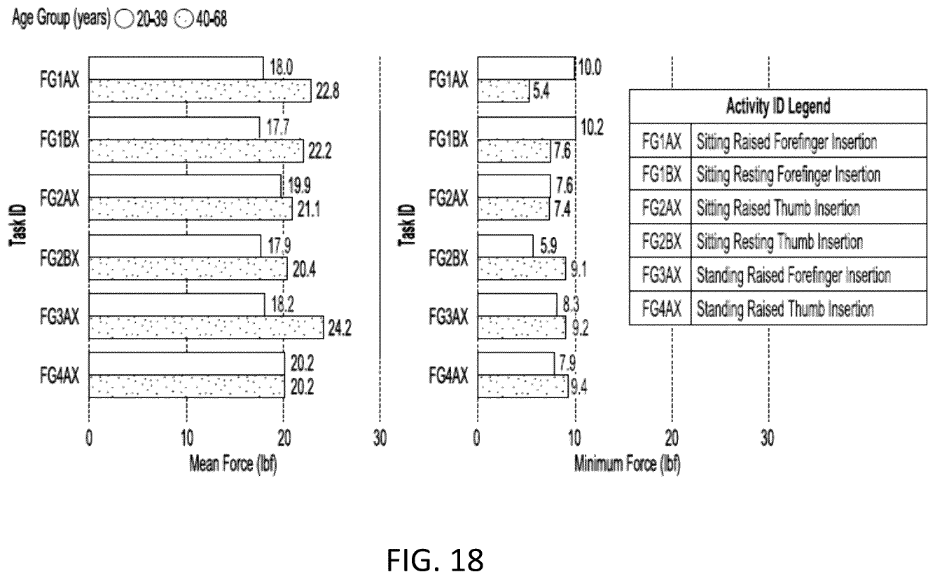

Further, as shown in FIG. 18, the study showed that participants aged 20-39 exert lower mean and minimum force than participants aged 40-69 for the majority of insertion tasks.

Regarding battery removal, Battery removal activities were conducted in six different body and arm positions, all using the participant's dominant hand. While seated, participants exerted maximum removal force using their forefinger and thumb as described below:
Forefingers, arms raised above the table (Activity FG1AR),
Forefingers, arms resting on the table (Activity FG1BR),
Thumb, arms raised above the table (Activity FG2AR),
Thumb, arms resting on the table (Activity FG2BR).

While standing, participants performed two additional removal tasks using their forefinger and thumb as described below:
Forefingers, arms raised in front of them (Activity FG3AR), and
Thumb, arms raised in front of them (Activity FG4AR).

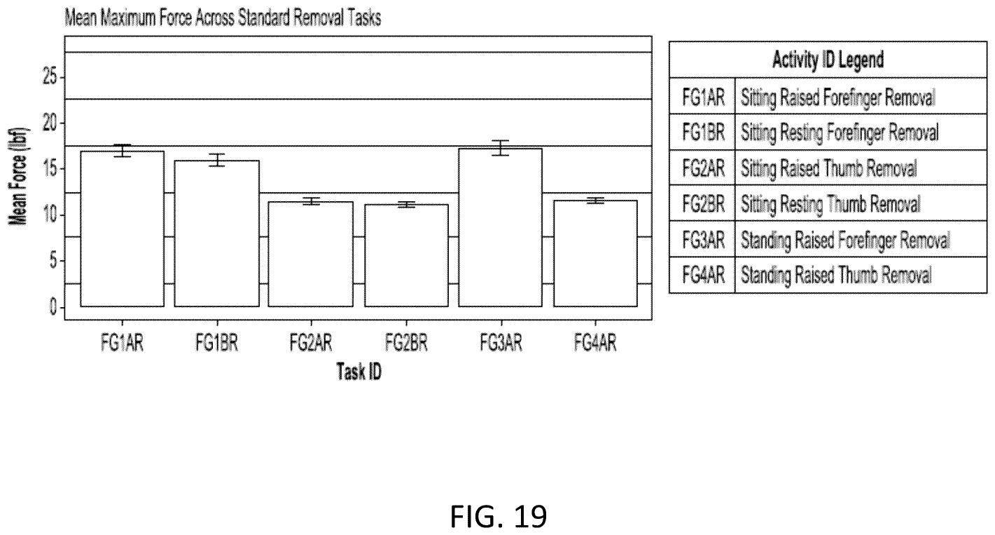

The mean maximum forces generated by participants for standard (forefinger and thumb) removal activity can be seen in FIG. 19. No additional force values were recorded for preference tasks, as all participants preferred one of the standard removal techniques. Seventeen participants preferred using their forefingers for battery removal and eight participants preferred thumb removal.

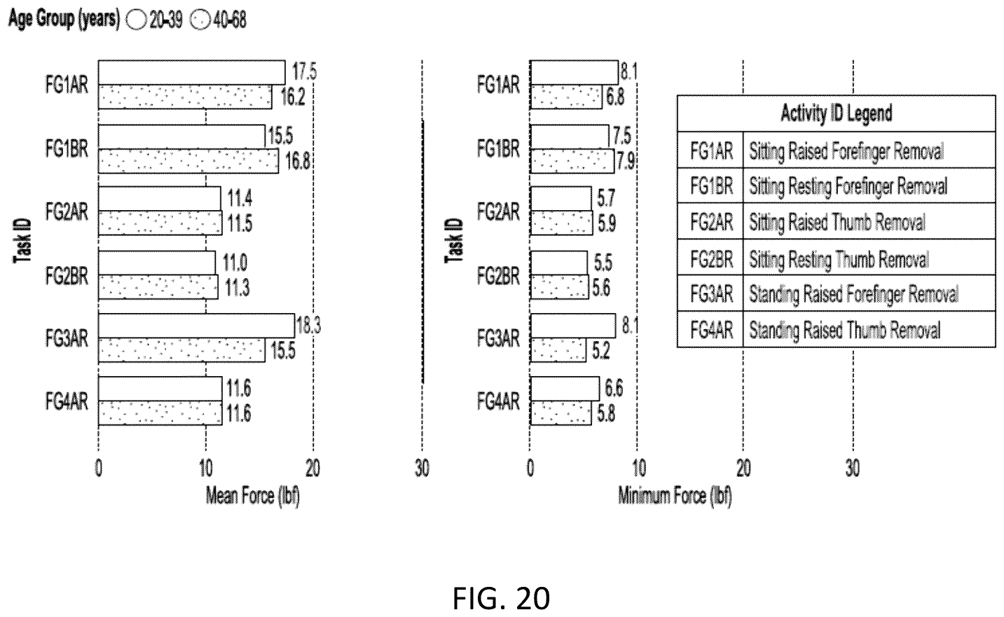

Further, as shown in FIG. 20, the study showed that participants aged 20-39 and aged 40-69 exerted similar mean and minimum maximum exerted force values for the majority of the battery removal tasks.

Participants in this study expressed positive opinions when interacting with the systems, devices, and techniques described herein. Further, the amount of force, tactile, and audio feedback were all mentioned positively. Of the 17 participants for whom palm insertion was their preferred method, 11 participants mentioned that the palm insertion felt easier and more natural to them. One participant found the battery and monitor easy to pick up and interact with. One participant mentioned that the force required to insert and remove the monitor was “just right”, and that they could interact with it all day. Two participants mentioned that the WCD monitor/controller itself was easy to pick up and understand how to insert and remove the battery without prior instruction.

Arm and body positions had no effect on the participants' ability to exert force during insertion or removal tasks. Mean insertion force across all standard tasks was similar, falling within a 1.5-lbf range. Palm insertion was most cited as a preferred insertion method, and palm insertion force was significantly greater than the insertion force applied using the forefingers. From observations, the force applied via the palm allowed for more efficient use of arm strength, rather than hand and finger strength in the case of the forefinger/thumb insertions. Participants exerted significantly greater force during forefinger removal tasks compared with thumb removal tasks. There existed a difference of ˜6 pounds of force between thumb and forefinger removal, with forefinger removal having an average of 18.4 lbf applied and thumb removal only 12.4 lbf. This may be a result of the forefingers using more surface area to grip onto and apply force to the battery latch than a single thumb, assuming that any combination of multiple forefingers will be able to work in tandem to exert greater force than a singular finger.

The teachings of the present disclosure can be generally applied to external medical monitoring and/or treatment devices that are powered by a battery. Such external medical devices can include, for example, ambulatory medical devices as described herein that are capable of and designed for moving with the patient as the patient goes about his or her daily routine. An example ambulatory medical device can be a wearable medical device such as a WCD, a wearable cardiac monitoring device, an in-hospital device such as an in-hospital wearable defibrillator (HWD), a short-term wearable cardiac monitoring and/or therapeutic device, mobile cardiac event monitoring devices, and other similar wearable medical devices.

The wearable medical device can be capable of continuous use by the patient. In some implementations, the continuous use can be substantially or nearly continuous in nature. That is, the wearable medical device can be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless be considered continuous use. For example, the wearable medical device can be configured to be worn by a patient for as many as 24 hours a day. In some implementations, the patient can remove the wearable medical device for a short portion of the day (e.g., for half an hour to bathe).

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other HCP provides specific instruction to the patient to stop use of the wearable medical device.

Regardless of the extended period of wear, the use of the wearable medical device can include continuous or nearly continuous wear by the patient as described above. For example, the continuous use can include continuous wear or attachment of the wearable medical device to the patient, e.g., through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (e.g., ECG information, including arrhythmia information, cardio-vibrations, etc.) and/or non-cardiac information (e.g., blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

As noted above, the wearable medical device can be configured to monitor other physiologic parameters of the patient in addition to cardiac related parameters. For example, the wearable medical device can be configured to monitor, for example, pulmonary-vibrations (e.g., using microphones and/or accelerometers), breath vibrations, sleep related parameters (e.g., snoring, sleep apnea), tissue fluids (e.g., using radio-frequency transmitters and sensors), among others.

Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, e.g., capable of providing therapeutic pacing pulses to the patient. In some examples, the ambulatory medical devices can be configured to monitor for and/or measure ECG metrics including, for example, heart rate (such as average, median, mode, or other statistical measure of the heart rate, and/or maximum, minimum, resting, pre-exercise, and post-exercise heart rate values and/or ranges), heart rate variability metrics, PVC burden or counts, atrial fibrillation burden metrics, pauses, heart rate turbulence, QRS height, QRS width, changes in a size or shape of morphology of the ECG information, cosine R-T, artificial pacing, QT interval, QT variability, T wave width, T wave alternans, T-wave variability, and ST segment changes.

As noted above, FIG. 1 illustrates an example component-level view of a medical device controller or monitor 100 included in, for example, a wearable medical device. As further shown in FIG. 1, the therapy delivery circuitry 102 can be coupled to one or more electrodes 120 configured to provide therapy to the patient. For example, the therapy delivery circuitry 102 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuitry and under control of one or more processors (e.g., processor 118) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). In some examples, the capacitors can include a single film or electrolytic capacitor as a series connected device including a bank of the same capacitors. These capacitors can be switched into a series connection during discharge for a defibrillation pulse. For example, a single capacitor of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 joules of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuitry 102 can be configured to perform the switching and pulse delivery operations, e.g., under control of the processor 118. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

In certain examples, the therapy delivery circuitry 102 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 104 can include one or more of non-transitory computer-readable media, such as flash memory, solid state memory, magnetic memory, optical memory, cache memory, combinations thereof, and others. The data storage 104 can be configured to store executable instructions and data used for operation of the medical device controller 100. In certain examples, the data storage can include executable instructions that, when executed, are configured to cause the processor 118 to perform one or more operations. In some examples, the data storage 104 can be configured to store information such as ECG data as received from, for example, the sensing electrode interface.

In some examples, the network interface 106 can facilitate the communication of information between the medical device controller 100 and one or more other devices or entities over a communications network. For example, where the medical device controller 100 is included in an ambulatory medical device, the network interface 106 can be configured to communicate with a remote computing device such as a remote server or other similar computing device. The network interface 106 can include communications circuitry for transmitting data in accordance with a Bluetooth® wireless standard for exchanging such data over short distances to an intermediary device. For example, such an intermediary device can be configured as a base station, a “hotspot” device, a smartphone, a tablet, a portable computing device, and/or other devices in proximity of the wearable medical device including the medical device controller 100. The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain examples, the user interface 108 can include one or more physical interface devices such as input devices, output devices, and combination input/output devices and a software stack configured to drive operation of the devices. These user interface elements can render visual, audio, and/or tactile content. Thus, the user interface 108 can receive input or provide output, thereby enabling a user to interact with the medical device controller 100.

The medical device controller 100 can also include at least one rechargeable battery 110 configured to provide power to one or more components integrated in the medical device controller 100. The rechargeable battery 110 can include a rechargeable multi-cell battery pack. In one example implementation, the rechargeable battery 110 can include three or more 2200 mAh lithium ion cells that provide electrical power to the other device components within the medical device controller 100. For example, the rechargeable battery 110 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 100.

The sensor interface 112 can include physiological signal circuitry that is coupled to one or more sensors configured to monitor one or more physiological parameters of the patient. As shown, the sensors can be coupled to the medical device controller 100 via a wired or wireless connection. The sensors can include one or more ECG sensing electrodes 122, and non-ECG physiological sensors 123 such as vibration sensor 124, tissue fluid monitors 126 (e.g., based on ultra-wide band radiofrequency devices), and motion sensors (e.g., accelerometers, gyroscopes, and/or magnetometers). In some implementations, the sensors can include a plurality of conventional ECG sensing electrodes in addition to digital sensing electrodes.

The sensing electrodes 122 can be configured to monitor a patient's ECG information. For example, by design, the digital sensing electrodes 122 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag-AgCl.

In some examples, the electrodes 122 can be used with an electrolytic gel dispersed between the electrode surface and the patient's skin. In certain implementations, the electrodes 122 can be dry electrodes that do not need an electrolytic material. As an example, such a dry electrode can be based on tantalum metal and having a tantalum pentoxide coating as is described above. Such dry electrodes can be more comfortable for long term monitoring applications.

Referring back to FIG. 1, the vibration sensors 124 be configured to detect cardiac or pulmonary vibration information. For example, the vibration sensors 124 can detect a patient's heart valve vibration information. For example, the vibration sensors 124 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 124 can also be configured to detect heart wall motion, for instance, by placement of the sensor in the region of the apical beat. The vibration sensors 124 can include a vibrational sensor configured to detect vibrations from a subject's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 124 can also include a multi-channel accelerometer, for example, a three-channel accelerometer configured to sense movement in each of three orthogonal axes such that patient movement/body position can be detected and correlated to detected cardio-vibrations information. The vibration sensors 124 can transmit information descriptive of the cardio-vibrations information to the sensor interface 112 for subsequent analysis.

The tissue fluid monitors 126 can use radio frequency (RF) based techniques to assess fluid levels and accumulation in a patient's body tissue. For example, the tissue fluid monitors 126 can be configured to measure fluid content in the lungs, typically for diagnosis and follow-up of pulmonary edema or lung congestion in heart failure patients. The tissue fluid monitors 126 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 126 can transmit information descriptive of the tissue fluid levels to the sensor interface 112 for subsequent analysis.

In certain implementations, the cardiac event detector 116 can be configured to monitor a patient's ECG signal for an occurrence of a cardiac event such as an arrhythmia or other similar cardiac event. The cardiac event detector can be configured to operate in concert with the processor 118 to execute one or more methods that process received ECG signals from, for example, the sensing electrodes 122 and determine the likelihood that a patient is experiencing a cardiac event. The cardiac event detector 116 can be implemented using hardware or a combination of hardware and software. For instance, in some examples, cardiac event detector 116 can be implemented as a software component that is stored within the data storage 104 and executed by the processor 118. In this example, the instructions included in the cardiac event detector 116 can cause the processor 118 to perform one or more methods for analyzing a received ECG signal to determine whether an adverse cardiac event is occurring. In other examples, the cardiac event detector 116 can be an application-specific integrated circuit (ASIC) that is coupled to the processor 118 and configured to monitor ECG signals for adverse cardiac event occurrences. Thus, examples of the cardiac event detector 116 are not limited to a particular hardware or software implementation.

In some implementations, the processor 118 includes one or more processors (or one or more processor cores) that each are configured to perform a series of instructions that result in manipulated data and/or control the operation of the other components of the medical device controller 100. In some implementations, when executing a specific process (e.g., cardiac monitoring), the processor 118 can be configured to make specific logic-based determinations based on input data received and be further configured to provide one or more outputs that can be used to control or otherwise inform subsequent processing to be carried out by the processor 118 and/or other processors or circuitry with which processor 118 is communicatively coupled. Thus, the processor 118 reacts to specific input stimulus in a specific way and generates a corresponding output based on that input stimulus. In some example cases, the processor 118 can proceed through a sequence of logical transitions in which various internal register states and/or other bit cell states internal or external to the processor 118 can be set to logic high or logic low. As referred to herein, the processor 118 can be configured to execute a function where software is stored in a data store coupled to the processor 118, the software being configured to cause the processor 118 to proceed through a sequence of various logic decisions that result in the function being executed. The various components that are described herein as being executable by the processor 118 can be implemented in various forms of specialized hardware, software, or a combination thereof. For example, the processor 118 can be a digital signal processor (DSP) such as a 24-bit DSP. The processor 118 can be a multi-core processor, e.g., having two or more processing cores. The processor 118 can be an Advanced RISC Machine (ARM) processor such as a 32-bit ARM processor or a 64-bit ARM processor. The processor 118 can execute an embedded operating system, and include services provided by the operating system that can be used for file system manipulation, display & audio generation, basic networking, firewalling, data encryption and communications.

As noted above, an ambulatory medical device such as a WCD can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 118 of the controller 100 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

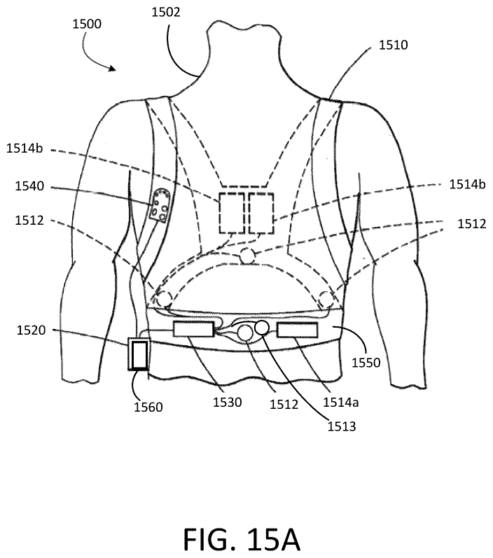

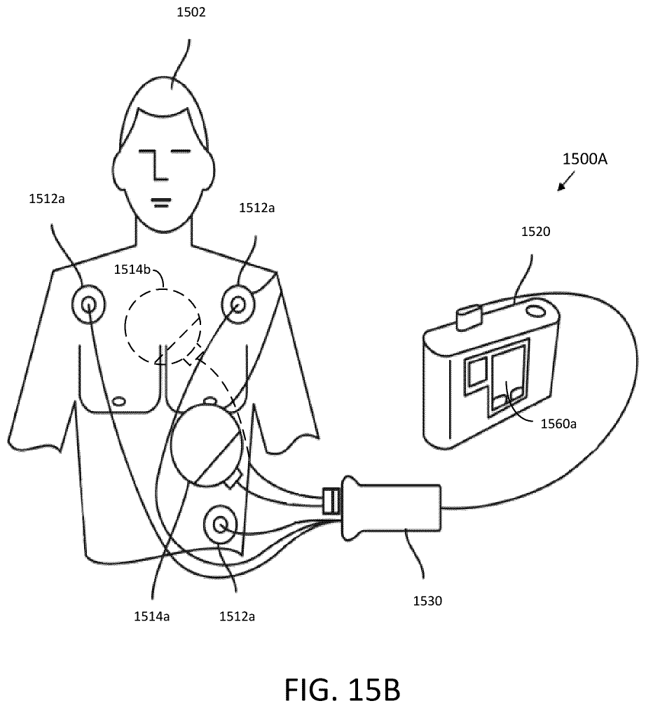

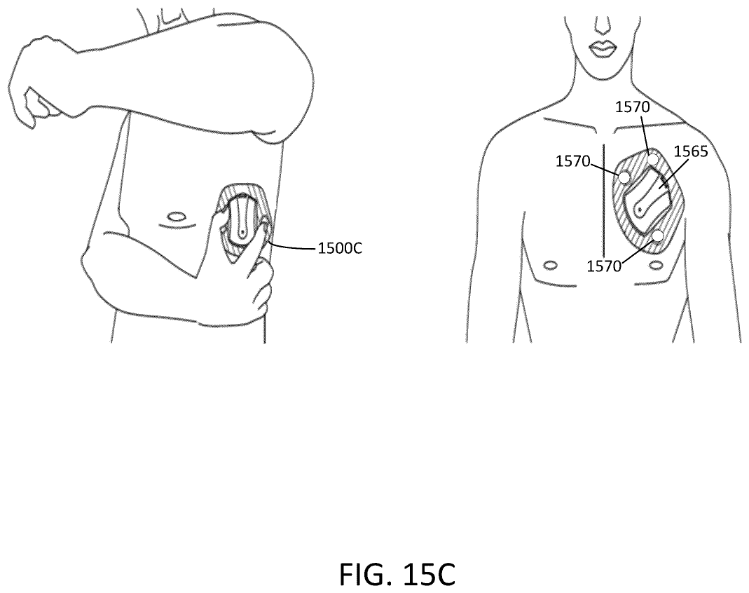

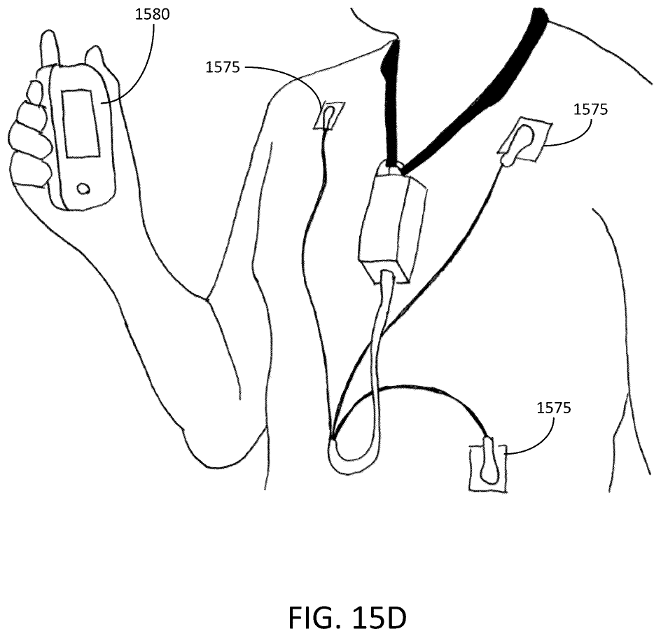

FIG. 15A illustrates an example medical device 1500 that is external, ambulatory, and wearable by a patient 1502, and configured to implement one or more configurations described herein. For example, the medical device 1500 can be a non-invasive medical device configured to be located substantially external to the patient. Such a medical device 1500 can be, for example, an ambulatory medical device that is capable of and designed for moving with the patient as the patient goes about his or her daily routine. For example, the medical device 1500 as described herein can be bodily-attached to the patient such as the LifeVest® wearable cardioverter defibrillator available from ZOLL® Medical Corporation. Such wearable defibrillators typically are worn nearly continuously or substantially continuously for two to three months at a time. During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 1500 can include one or more of the following: a garment 1510, one or more ECG sensing electrodes 1512, one or more non-ECG physiological sensors 1513, one or more therapy electrodes 1514a and 1514b (collectively referred to herein as therapy electrodes 1514), a medical device controller 1520 (e.g., controller 100 as described above in the discussion of FIG. 1), a connection pod 1530, a patient interface pod 1540, a belt 1550, or any combination of these. In some examples, at least some of the components of the medical device 1500 can be configured to be affixed to the garment 1510 (or in some examples, permanently integrated into the garment 1510), which can be worn about the patient's torso.

The medical device controller 1520 can be operatively coupled to the sensing electrodes 1512, which can be affixed to the garment 1510, e.g., assembled into the garment 1510 or removably attached to the garment, e.g., using hook and loop fasteners. In some implementations, the sensing electrodes 1512 can be permanently integrated into the garment 1510. The medical device controller 1520 can be operatively coupled to the therapy electrodes 1514. For example, the therapy electrodes 1514 can also be assembled into the garment 1510, or, in some implementations, the therapy electrodes 1514 can be permanently integrated into the garment 1510. In an example, the medical device controller 1520 includes a patient user interface 1560 to allow a patient interface with the externally-worn device. For example, the patient can use the patient user interface 1560 to respond to pre-and post-workout questions, prompts, and surveys as described herein.

Component configurations other than those shown in FIG. 15A are possible. For example, the sensing electrodes 1512 can be configured to be attached at various positions about the body of the patient 1502. The sensing electrodes 1512 can be operatively coupled to the medical device controller 1520 through the connection pod 1530. In some implementations, the sensing electrodes 1512 can be adhesively attached to the patient 1502. In some implementations, the sensing electrodes 1512 and at least one of the therapy electrodes 1514 can be included on a single integrated patch and adhesively applied to the patient's body.

The sensing electrodes 1512 can be configured to detect one or more cardiac signals. Examples of such signals include ECG signals and/or other sensed cardiac physiological signals from the patient. In certain examples, as described herein, the non-ECG physiological sensors 1513 such as accelerometers, vibrational sensors, and other measuring devices for recording additional non-ECG physiological parameters. For example, as described above, the such non-ECG physiological sensors are configured to detect other types of patient physiological parameters and acoustic signals, such as tissue fluid levels, cardio-vibrations, lung vibrations, respiration vibrations, patient movement, etc.

In some examples, the therapy electrodes 1514 can also be configured to include sensors configured to detect ECG signals as well as other physiological signals of the patient. The connection pod 1530 can, in some examples, include a signal processor configured to amplify, filter, and digitize these cardiac signals prior to transmitting the cardiac signals to the medical device controller 1520. One or more of the therapy electrodes 1514 can be configured to deliver one or more therapeutic defibrillating shocks to the body of the patient 1502 when the medical device 1500 determines that such treatment is warranted based on the signals detected by the sensing electrodes 1512 and processed by the medical device controller 1520. Example therapy electrodes 1514 can include metal electrodes such as stainless-steel electrodes that include one or more conductive gel deployment devices configured to deliver conductive gel to the metal electrode prior to delivery of a therapeutic shock.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 1514 and associated circuitry can be optionally decoupled from (or coupled to) or switched out of (or switched in to) the medical device. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 15B illustrates a hospital wearable defibrillator 1500A that is external, ambulatory, and wearable by a patient 1502. Hospital wearable defibrillator 1500A can be configured in some implementations to provide pacing therapy, e.g., to treat bradycardia, tachycardia, and asystole conditions. The hospital wearable defibrillator 1500A can include one or more ECG sensing electrodes 1512a, one or more therapy electrodes 1514a and 1514b, a medical device controller 1520 and a connection pod 1530. For example, each of these components can be structured and function as like number components of the medical device 1500. For example, the electrodes 1512a, 1514a, 1514b can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 1514a attaches to the front of the patient's torso to deliver pacing or defibrillating therapy. Similarly, the back adhesively attachable therapy electrode 1514b attaches to the back of the patient's torso. In an example scenario, at least three ECG adhesively attachable sensing electrodes 1512a can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 1560a can be configured to interact with a user other than the patient, e.g., a nurse, for device-related functions such as initial device baselining, setting and adjusting patient parameters, and changing the device batteries.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 15A.

FIGS. 15C and 15D illustrate example wearable patient monitoring devices with no treatment or therapy functions. For example, such devices are configured to monitor one or more physiological parameters of a patient, e.g., for remotely monitoring and/or diagnosing a condition of the patient. For example, such physiological parameters can include a patient's ECG information, tissue (e.g., lung) fluid levels, cardio-vibrations (e.g., using accelerometers or microphones), and other related cardiac information. A cardiac monitoring device is a portable device that the patient can carry around as he or she goes about their daily routine.

Referring to FIG. 15C, an example wearable patient monitoring device 1500C can include tissue fluid monitors 1565 that use radio frequency (RF) based techniques to assess fluid levels and accumulation in a patient's body tissue. Such tissue fluid monitors 1565 can be configured to measure fluid content in the lungs, typically for diagnosis and follow-up of pulmonary edema or lung congestion in heart failure patients. The tissue fluid monitors 1565 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 1500C may be a cardiac monitoring device that also includes digital sensing electrodes 1570 for sensing ECG activity of the patient. Device 1500C can pre-process the ECG signals via one or more ECG processing and/or conditioning circuits such as an ADC, operational amplifiers, digital filters, signal amplifiers under control of a microprocessor. Device 1500C can transmit information descriptive of the ECG activity and/or tissue fluid levels via a network interface to a remote server for analysis.

Referring to FIG. 15D, another example wearable cardiac monitoring device 1500D can be attached to a patient via at least three adhesive digital cardiac sensing electrodes 1575 disposed about the patient's torso. Cardiac devices 1500C and 1500D are used in cardiac monitoring and telemetry and/or continuous cardiac event monitoring applications, e.g., in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring for an extended period of time, e.g., 10 to 30 days, or more. In some ambulatory cardiac monitoring and/or telemetry applications, a portable cardiac monitoring device can be configured to substantially continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heart rate, fatigue, fainting, chest discomfort, weakness, dizziness, and/or giddiness. The cardiac monitoring device can record predetermined physiologic parameters of the patient (e.g., ECG information) for a predetermined amount of time (e.g., 1-30 minutes before and 1-30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (e.g., using accelerometers or microphones), pulmonary-vibrational signals, breath vibrations, sleep related parameters (e.g., snoring, sleep apnea), tissue fluids, among others.

In some examples, the devices described herein (e.g., FIGS. 15A-D) can communicate with a remote server via an intermediary device 1580 such as that shown in FIG. 15D. For instance, devices such as shown in FIGS. 15A-D can be configured to include a network interface communications capability as described herein in reference to, for example, FIG. 1.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the scope of the appended claims. For example, it is to be understood that the present disclosure contemplates that, to the extent possible, one or more features of any embodiment can be combined with one or more features of any other embodiment.

Other examples are within the scope of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1-45. (canceled)

46. A system for providing battery insertion feedback to patients wearing or using a wearable cardioversion-defibrillation device, the system comprising:
a controller of the wearable cardioversion-defibrillation device;
a battery well disposed on the controller;
a removable battery configured to be inserted into the battery well such that an electrical connection is established between the removable battery and the controller;
a mechanical attachment disposed on either the removable battery or within an interior volume of the battery well, the mechanical attachment configured at least to detect insertion of the removable battery within the battery well; and
a visual indicator operably coupled to the mechanical attachment, the visual indicator configured to provide visual feedback to a patient using the wearable cardioversion-defibrillation device that the removable battery is inserted into the battery well and providing power to the wearable cardioversion-defibrillation device to monitor and/or treat the patient.

47. The system of claim 46, wherein the mechanical attachment is further configured to exert an opposition force upon the removable battery as the removable battery is inserted within the battery well.

48. The system of claim 47, wherein the mechanical attachment is further configured such that, upon release of the removable battery from the battery well, the opposition force causes at least partial ejection of the removable battery from the battery well.

49. The system of claim 48, wherein the visual indicator is configured to alter the visual feedback in response to changes in the opposition force exerted by the mechanical attachment as the removable battery is inserted within the battery well.

50. The system of claim 46, wherein the mechanical attachment comprises at least one movable pawl operably coupled to the visual indicator, the movable pawl configured to be displaced from within an interior volume of the battery well as the removable battery is inserted into the battery well.

51. The system of claim 50, wherein movement of the movable pawl causes movement of at least a portion of the visual indicator, thereby changing the visual feedback provided to the patient.

52. The system of claim 46, further comprising a battery latching mechanism configured to secure the removable battery in the battery well and provide tactile feedback to the patient upon proper insertion of the removable battery within the battery well.

53. The system of claim 46, further comprising battery circuitry disposed in either the removable battery or within the battery well, the battery circuitry configured to detect whether the removable battery is inserted into the battery well and providing power to monitor and/or treat the patient.

54. The system of claim 53, wherein the battery circuitry comprises at least one feedback device configured to output one or more of tactile, visual, or audio feedback.

55. The system of claim 53, wherein the battery circuitry further comprises battery detection circuitry configured to detect a position of the removable battery within the battery well.

56. A wearable cardioversion-defibrillation system for providing battery information to patients, the system comprising:
one or more sensing electrodes configured to output a signal indicative of cardiac activity of a patient;
one or more therapy electrodes configured to provide one or more treatment shocks to the patient when a patient wearing the wearable cardioversion-defibrillation system experiences a cardiac arrhythmia;
a controller of the wearable cardioversion-defibrillation system, the controller operably coupled to the one or more sensing electrodes and the one or more therapy electrodes;
a battery well disposed on the controller; and
battery circuitry disposed in either a removable battery or within the battery well, the battery circuitry configured to
detect whether the removable battery is inserted into the battery well and providing power to monitor and/or treat the patient, and
provide human-perceptible confirmation via one or more of tactile, visual, or audio feedback to the patient on detecting that the removable battery has been inserted into the battery well and is providing power to monitor and/or treat the patient;
wherein upon proper insertion of the removable battery within the battery well, the controller is configured to monitor and/or treat the patient for a cardiac arrhythmia based on power from the removable battery,
wherein the battery well comprises an electrical connector configured to receive at least a portion of the removable battery to establish an electrical connection between the removable battery and the controller, and
wherein the battery well further comprises a seal configured to at least partially deform when the removable battery is inserted into the battery well to securely enclose the electrical connection between the removable battery and the controller.

57. The system of claim 56, wherein the battery circuitry comprises at least one feedback device configured to output the one or more tactile, visual, or audio feedback.

58. The system of claim 57, wherein the at least one feedback device comprises at least one visual indicator configured to transition from a first visual state to a second visual state upon proper insertion of the removable battery into the battery well.

59. The system of claim 57, wherein the at least one feedback device comprises at least one audio output device configured to output the audio feedback upon proper insertion of the removable battery into the battery well.

60. The system of claim 57, wherein the at least one feedback device comprises a vibrational mechanism configured to output the tactile feedback when the removable battery is inserted into the battery well.

61. The system of claim 57, wherein the removable battery comprises a housing and the at least one feedback device is integrated into the housing of the removable battery.

62. A wearable cardioversion-defibrillation system for providing battery information to patients, the system comprising:
one or more sensing electrodes configured to output a signal indicative of cardiac activity of a patient;
one or more therapy electrodes configured to provide one or more treatment shocks to the patient when a patient wearing the wearable cardioversion-defibrillation system experiences a cardiac arrhythmia;
a controller of the wearable cardioversion-defibrillation system, the controller operably coupled to the one or more sensing electrodes and the one or more therapy electrodes;
a battery well disposed on the controller; and
battery circuitry disposed in either a removable battery or within the battery well, the battery circuitry configured to detect whether the removable battery is inserted into the battery well and providing power to monitor and/or treat the patient,
wherein upon proper insertion of the removable battery within the battery well, the controller is configured to monitor and/or treat the patient for a cardiac arrhythmia based on power from the removable battery, and
wherein the battery circuitry comprises at least one feedback device comprising at least one visual indicator configured to transition from a first visual state to a second visual state upon proper insertion of the removable battery into the battery well.

63. The system of claim 62, wherein the at least one feedback device comprises at least one audio output device configured to output audio feedback upon proper insertion of the removable battery into the battery well.

64. The system of claim 63, wherein the at least one audio output device is adjustable to alter a predetermined frequency range of the audio feedback.

65. The system of claim 62, wherein the at least one feedback device further comprises a vibrational mechanism configured to output tactile feedback when the removable battery is inserted into the battery well.

66. The system of claim 62, wherein the removable battery comprises a housing and the at least one feedback device is integrated into the housing of the removable battery.

67. The system of claim 62, further comprising a battery latching mechanism configured to secure the removable battery in the battery well and provide additional tactile feedback to the patient upon proper insertion of the removable battery within the battery well.

68. The system of claim 62, wherein the battery circuitry further comprises battery detection circuitry configured to detect a position of the removable battery within the battery well.

69. The system of claim 62, further comprising a battery charger, the battery charger comprising a charging battery well, the charging battery well comprising a charging connector.
